(19) United States
(12) Patent Application Publication
(10) Pub. No.: US 2022/0049260 A1
(43) Pub. Date: Feb. 17, 2022

(54) INDUCIBLE EXPRESSION SYSTEM FOR PLASMID-FREE PRODUCTION OF A PROTEIN OF INTEREST

(71) Applicant: Boehringer Ingelheim RCV GmbH & Co. KG, Ingelheim am Rhein (DE)

(72) Inventors: Artur SCHULLER, Vienna (AT); Monika CSERJAN, Langenzersdorf (AT); Reingard GRABHERR, Vienna (AT); Gerald STRIEDNER, Vienna (AT)

(21) Appl. No.: 17/274,799

(22) PCT Filed: Sep. 11, 2019
(86) PCT No.: PCT/EP2019/074239

§ 371 Date: Mar. 10, 2021

(30) Foreign Application Priority Data
Sep. 11, 2018 (EP) 18193655.0

(51) Int. Cl. C12N 15/72

(52) U.S. Cl. CPC C12N 15/72

(57) ABSTRACT

A genome-based expression system for production of a protein of interest (POI) in a prokaryotic host, comprising at least an RNA polymerase (RNAP) gene, a gene encoding a POI, comprising a coding sequence, a promoter operably linked to said coding sequence, wherein said promoter is recognized by the RNAP expressed from the RNAP gene, and at least one lac operator (lacO) within the sequence of said promoter; and a lad gene encoding a lac repressor protein (LacI) comprising a coding sequence, a lacI promoter operably linked to the lad coding sequence, wherein the lacI promoter is a wild-type lacI promoter or a lacI promoter which increases LacI expression; wherein the expression rate of the POI is regulated by an inducer binding LacI.

### FIELD OF THE INVENTION

The invention relates to the field of plasmid-free inducible systems for expression of a protein of interest in a prokaryotic host. It further relates to methods of using such systems for the production of a protein of interest in a prokaryotic host.

### BACKGROUND OF THE INVENTION

In industrial protein production processes, gene regulation is an important prerequisite. Transcription rates are controlled by the interaction of a promoter and the RNA polymerase (RNAP). Understanding and external regulation of this interaction is necessary to provide process control and optimization of product yield and quality. A reduced promoter strength can be beneficial, especially for challenging proteins, like antibody fragments, membrane proteins or toxic proteins (1-3). The final product yield of soluble and proper folded proteins is often not directly determined by the strength of the promoter system but by further processing of the peptide chains, like translocation into the periplasm and proper disulfide bond formation.

The most prominent and well-studied genetic regulatory mechanism is the lac operon (4). In wild-type E. coli, the lac-inhibitor (LacI) forms a homo-tetramer that binds to the lac-operator sequences (lacO) and represses the transcription of the IacZYA operon (5). In the presence of lactose or the non-metabolizable isopropyl β-D-1-thiogalactopyranoside (IPTG), LacI changes in structure and can no longer bind to the lac-operator, resulting in induction of transcription. The lac-operator sites are DNA sequences with inverted repeat symmetry (6).

The higher the symmetry, the greater the binding affinity of LacI to the operator sequence. An artificial perfectly symmetric lacO (sym-lacO) was found to bind LacI with the greatest affinity (7), whereas the three wild-type operators lacO1, lacO2 and lacO3 exhibiting an approximate symmetry showed lower affinities, resulting in the following order: sym-lacO>lacO1>lacO2>lacO3 (8). LacI binds simultaneously to both, the primary operator lacO1 and to either lacO2 or lacO3 through a DNA-looping mechanism (9). LacO2 is located 401 bp downstream of lacO1, whereas lacO3 lies only 92 bp upstream of lacO1 (10). The role of lacO2 is still not clear, because the main contribution to repression comes from the DNA-looping of lacO1 and lacO3 due to their closer proximity (8). Furthermore, when lacO1 and lacO3 are bound by LacI, the production of LacI itself is prevented. The 3′ end of the lacI gene overlaps with lacO3. In a repressed state, transcription of lacI results in a truncated mRNA, which is rapidly degraded by the cell. Due to this autoregulation, the concentration of the LacI tetramer is ˜40 molecules in induced cells and ˜15 molecules in non-induced cells (11).

Several mutants of the LacI repressor protein and the pLacI promoter exist. Penumetcha et al. tested various combinations of repressor and promoter mutants in an effort to discover a system with reduced leakiness in transcription. They report that use of the wild-type LacI repressor protein in combination with the pLacI^{Q1} Promoter gives high levels of induction and low levels of leaky transcription (34).

Oehler et al. tested the effect of systematic destruction of all three lac operators of the chromosomal lac operon of Escherichia coli on repression by Lac repressor and report that the three operators of the lac operon cooperate in repression (35).

The tetrameric Lac repressor can bind simultaneously to two lac operators on the same DNA molecule, thereby including the formation of a DNA loop. Müller et al. report that repression increases significantly with decreasing inter-operator DNA length (36).

The effects of placing a lac operator at different positions relative to a promoter for bacteriophage T7 RNA polymerase have been tested. Transcription can be strongly repressed by lac repressor bound to an operator 15 base-pairs downstream from the RNA start (37).

WO2003/050240A2 discloses an expression system for producing a target protein in a host cell comprising a homologously integrated gene encoding T7 RNA polymerase, and a non-integrated gene encoding a target protein.

One of the first applications of the lac regulatory mechanism was the pET system, which today is the most widely used E. coli expression system for recombinant protein production (12, 13). This system is based on the specific interaction of the T7-phage derived T7 RNAP with the strong T7 promoter. The recombinase functions of bacteriophage lambda were used for site-directed insertion of the T7 RNA polymerase gene into the E. coli genome. Expression of the T7 RNAP is controlled by the lacUV5 promoter, a variant of the lactose promoter that is insensitive to catabolic repression. Addition of IPTG, induces the expression of the T7 RNAP at high levels, which in turn transcribes the target gene which is under control of the T7 promoter. This orthogonal expression system offers very high product titres for recombinant proteins that can efficiently be produced in E. coli. However, the extraordinary strength of the T7 expression system, especially if combined with high-copy number plasmids exerts an extreme metabolic load on the host cells. When the gene of interest codes for challenging proteins, stress and metabolic burden often lead to reduced yield, shortened production periods and even cell death (14, 15).

Plasmid-mediated stress effects, such as high gene dosage and transcription of antibiotic resistance genes, can be overcome by integration of the gene of interest (GOI), i.e. the gene encoding the protein of interest, into the host's genome (16, 17).

WO2008/142028A1 discloses a method for producing a protein of interest, wherein the DNA encoding the protein of interest is integrated into a bacterial cell's genome at a pre-selected site.

Striedner et al. disclose a plasmid-free T7 based Escherichia coli expression system, wherein the target gene is site-specifically integrated into the genome of the host (17).

Genome integrated T7-based expression systems offer significant advantages. Compared to plasmid-based expression systems there is no plasmid mediated metabolic load and no variation in gene dosage during the production process. However, the T7 RNA polymerase (RNAP) is prone to mutations under long-term production conditions. This was demonstrated by Striedner et al. (17) in chemostat cultivations, where mutations in the T7 RNAP led to faster growing of a non-producing cell population and thus, to a massive loss in product yield.

There is thus a clear need in the field for improved inducible expression systems which result in improved expression rates, low basal expression and true tunability of expression rates on a cellular level, even at low inductor concentrations.

### SUMMARY OF THE INVENTION

It is the objective of the present invention to provide an improved inducible system with improved control of expression rate of a protein of interest and very low basal expression for plasmid-free production of a protein of interest.

The problem is solved by the present invention.

According to the invention, there is provided a genome-based expression system for production of a protein of interest in a prokaryotic host, comprising at least

a) an RNA polymerase (RNAP) gene,

b) a gene for expression of a protein of interest, comprising
a coding sequence encoding the protein of interest,
a promoter operably linked to said coding sequence, wherein said promoter is recognized by the RNAP expressed from a), and
at least one lac operator (lacO) within the sequence of said promoter; and

c) a lacI gene for expression of a lac repressor protein (LacI) comprising
a lacI coding sequence,
a lacI promoter operably linked to the lacI coding sequence, wherein the lacI promoter is selected from the group consisting of wild-type lacI and a lacI promoter which increases lacI expression;

wherein the expression rate of the protein of interest is regulated by an inducer binding LacI.

According to a specific embodiment, there is provided a genome-based expression system for production of a protein of interest in a prokaryotic host, comprising at least
a) an RNA polymerase (RNAP) gene,
b) a gene for expression of a protein of interest, comprising
a coding sequence encoding the protein of interest,
a promoter operably linked to said coding sequence, wherein said promoter is recognized by the RNAP expressed from a), and
a lac operator (lacO), preferably lacO1, within the sequence of said promoter; and
c) a lacI gene for expression of a lac repressor protein (LacI) comprising
a lacI coding sequence
a lacI promoter operably linked to the lacI coding sequence, wherein the lacI promoter is a lacI promoter which increases expression of lacI, preferably it is the lacI^{Q} promoter;

wherein the expression rate of the protein of interest is regulated by an inducer binding LacI.

Specifically, the gene for expression of a protein of interest contains one lacO within the sequence of the promoter operably linked to the coding sequence, and the lacI promoter is a promoter which increases LacI expression.

According to a further specific embodiment, there is provided genome-based expression system for production of a protein of interest in a prokaryotic host, comprising at least

a) an RNA polymerase (RNAP) gene,

b) a gene for expression of a protein of interest, comprising
a coding sequence encoding the protein of interest,
a promoter operably linked to said coding sequence, wherein said promoter is recognized by the RNAP expressed from a), and
at least two lac operators (lacOs) that are at least 92 bp, specifically 94 bp, apart, wherein one lacO is within the sequence of the promoter and the other lacO is upstream of the promoter; and

c) a lacI gene for expression of a lac repressor protein (LacI) comprising
a lacI coding sequence
a lacI promoter operably linked to the lacI coding sequence, wherein the lacI promoter is the wild-type lacI promoter;

wherein the expression rate of the protein of interest is regulated by an inducer binding LacI.

According to an alternative embodiment, there is provided an inducible system for plasmid-free production of a protein of interest in a prokaryotic host, comprising at least

a) an RNA polymerase (RNAP) gene in the chromosome of the host,

b) a gene for expression of a protein of interest comprising
a coding sequence encoding the protein of interest,
a promoter operably linked to said coding sequence, wherein said promoter is recognized by the RNAP expressed from a), and
at least one lac operator (lacO) within the sequence of said promoter; and

c) a lacI gene for expression of a lac repressor protein (lacI) comprising
a lacI coding sequence,
a lacI promoter operably linked to the lacI coding sequence, wherein the lacI promoter is selected from the group consisting of wild-type lacI and a lacI promoter which increases expression of lad;

wherein the affinity of lacI to the one or more lacO/lacOs of b) is lower than the affinity of lacI to the lac operators lacO1 and lacO3 of the endogenous lac operon of the host and wherein the expression rate of the protein of interest is regulated by an inducer binding LacI.

According to further embodiment, there is provided an inducible system for plasmid-free production of a protein of interest in a prokaryotic host, comprising at least

a) an RNA polymerase (RNAP) gene in the chromosome of the host,

b) a gene for expression of a protein of interest comprising
a coding sequence encoding the protein of interest,
a promoter operably linked to said coding sequence, wherein said promoter is recognized by the RNAP expressed from a), and
a lac operator (lacO), preferably lacO1, within the sequence of said promoter; and

c) a lacI gene for expression of a lac repressor protein (lacI) comprising
a lacI coding sequence
a lacI promoter operably linked to the lacI coding sequence, wherein the lacI promoter is a lacI promoter which increases expression of lacI, preferably it is the lacI^{Q} promoter;

wherein the affinity of lacI to the one lacO of b) is lower than the affinity of lacI to the lac operators lacO1 and lacO3 of the endogenous lac operon of the host and wherein the expression rate of the protein of interest is regulated by an inducer binding LacI.

According to a further specific embodiment of the invention, there is provided an inducible system for plasmid-free production of a protein of interest in a prokaryotic host, comprising at least

a) an RNA polymerase (RNAP) gene in the chromosome of the host,

b) a gene for expression of a protein of interest comprising
a coding sequence encoding the protein of interest,
a promoter operably linked to said coding sequence, wherein said promoter is recognized by the RNAP expressed from a), and
at least two lac operators (lacOs) that are at least 92 bp apart, wherein one lacO is within the sequence of the promoter and the other lacO is upstream of the promoter; and

c) a lacI gene for expression of a lac repressor protein (lacI) comprising
a lacI coding sequence
a lacI promoter operably linked to the lacI coding sequence, wherein the lacI promoter is the wild-type lacI promoter;

wherein the affinity of lacI to the at least two lacOs of b) is lower than the affinity of lacI to the lac operators lacO1 and lacO3 of the endogenous lac operon of the host and wherein the expression rate of the protein of interest is regulated by an inducer binding LacI.

Specifically, the prokaryotic host is Escherichia coli (E. coli). Specifically, the host is E. coli of the strain BL21 or K-12.

Specifically, the RNAP is an RNAP homologous to the host, specifically a⁷° E. coli RNA polymerase.

Specifically, the promoter operably linked to the coding sequence encoding the protein of interest is selected from the group consisting of T5, T5N25, T7A1, T7A2, T7A3, lac, lacUV5, tac or trc or functional variants thereof with at least 20, 30, 40, 50, 60, 70, 80 or 90% sequence identity to T5, T7A1, T7A2, T7A3, lac, lacUV5, tac or trc.

According to a preferred embodiment of the inducible system described herein, the lacI promoter is a promoter which increases expression of LacI compared to the wild type host, which is the lacI^{Q} promoter (SEQ ID NO:1). Specifically, the gene encoding the protein of interest includes only one lacO, preferably lacO1, and the lacI promoter is lacI^{Q} (SEQ ID NO:1).

Preferably, the gene encoding the protein of interest comprises at least one lacO selected from the group consisting of lacO1, lacO2 or lacO3 and any combination thereof. Specifically, the gene encoding the protein of interest comprises two lacOs, preferably lacO1 and lacO1 or lacO1 and lacO2 or lacO1 and lacO3.

Specifically, the at least one lac operator comprised in the gene encoding the protein of interest is a lacO1 (SEQ ID NO:3), lacO2 (SEQ ID NO:4) or lacO3 (SEQ ID NO:5).

Specifically, the at least one lac operator is a functional variant of lacO1, lacO2 or lacO3 with at least 65% sequence identity or a perfectly symmetric lacO. Specifically, the lac operator is a functional variant of lacO1, lacO2 or lacO3 with at least 66, 67, 68, 69, 70, 71, 72, 73, 74, 75, 76, 77, 78, 79, 80, 81, 82, 83, 84, 85, 86, 87, 88, 89, 90, 91 or 95% sequence identity to wild-type lacO1, lacO2 or lacO3. According to an alternative, a functional variant of lacO1, lacO2 or lacO3 comprises 1, 2, 3, 4 or 5 point mutations or deletions of 1, 2, 3, 4 or 5 base pairs (bps).

Specifically, said promoter operably linked to the coding sequence encoding the protein of interest comprises an initial transcribed sequence (ITS), preferably a native T7A1 initial transcribed sequence (SEQ ID NO:2).

According to the system provided herein, the expression rate of the protein of interest is regulated by an inducer binding LacI. Specifically, LacI binds to the at least one lacO thereby repressing transcription of the gene encoding the protein of interest. Specifically, upon addition of an inducer capable of binding LacI interaction of LacI with the at least one lacO is prevented, resulting in induction of transcription of the gene encoding the protein of interest.

Specifically, the inducer is selected from the group consisting of isopropylthiogalactoside (IPTG), lactose, methyl-β-D-thiogalactoside, phenyl-β-D-galactose and ortho-Nitrophenyl-β-galactoside (ONPG).

Specifically, the promoter operably linked to the coding sequence expressing the protein of interest comprises an initial transcribed sequence, preferably the native T7A1 initial transcribed sequence. Specifically, the initial transcribed sequence is not limited to the ITS of T7A1 and can be any ITS known to a person skilled in the art.

According to a specific embodiment of the inducible system provided herein, the gene for expression of a protein of interest contains one lacO1 operator within the sequence of the promoter operably linked to the native T7A1 initial transcribed sequence (SEQ ID NO:2) and to the coding sequence, and wherein the LacI promoter is a lacI^{Q} promoter.

According to a further specific embodiment of the inducible system provided herein, the gene of interest contains two lac operators which are at least about 92 or 94 basepairs (bps) apart, preferably at least about 103, 105, 114, 116, 125, 127, 136, 138, or 149 bps apart, wherein one lac operator is located within the sequence of the promoter operably linked to the coding sequence and the second lac operator is upstream of the promoter.

Specifically, the gene encoding the protein of interest is a heterologous gene. Specifically, said gene that is heterologous to the prokaryotic host is a recombinant gene that is introduced into the host.

According to a further specific embodiment, the gene encoding the protein of interest is a homologous gene. Specifically, said gene that is homologous to the prokaryotic host, comprises a coding sequence, encoding the protein of interest, a promoter operably linked to said coding sequence, wherein said promoter is recognized by an RNAP that is expressed from a gene in the chromosome of the host, and at least one lac operator (lacO) within the sequence of said promoter.

Specifically, said gene that is homologous to the prokaryotic host is a recombinant gene that is introduced into the host. According to yet a further specific embodiment, said gene that is homologous to the prokaryotic host is modified by replacement of the promoter endogenous to said gene with a promoter described herein. Replacement can also mean the integration of the promoter described herein so that it is operably linked to the endogenous homologous gene/polypeptide in the chromosome/genome of the host cell wherein the naturally occurring promoter of the endogenous homologous gene/polypeptide is inactivated by at least one point mutation within the naturally occurring promoter. Specifically, the promoter endogenous to said gene is replaced with a promoter described herein comprising at least one lacO within the sequence of the promoter, preferably at least two lacOs, wherein one lacO is within the sequence of the promoter and a second lacO is upstream of the promoter. Specifically, the affinity of lacI to the one or more lacO/lacOs of the promoter replacing the endogenous promoter of the gene encoding the protein of interest is lower than the affinity of LacI to the lacO1 and lacO3 of the endogenous lac operon.

Specifically, the promoter operably linked to the coding sequence of the gene for expression of a protein of interest, is a recombinant promoter. Specifically, said promoter is not the wildtype lac promoter, it can, however, be a variant of the lac promoter. In the case, where the promoter described herein is a variant of the lac promoter, it comprises at least one lacO within its sequence, specifically it comprises at least one lacO within the sequence between the −10 and −35 promoter elements.

Further provided herein is a method of plasmid-free production of a protein of interest in a prokaryotic host, using the inducible system described herein, comprising the steps of

a) cultivating the host cells and inducing expression of the gene of interest by addition of an inducer,

b) harvesting the protein of interest, and

c) isolating and purifying the protein of interest and optionally

d) modifying the protein of interest and

e) formulating the protein of interest.

According to a specific embodiment of the system described herein, the gene for producing the protein of interest and/or the lacI gene for producing a lac repressor protein are comprised in at least one expression cassette. Preferably, said expression cassette is used to integrate the gene for producing the protein of interest and/or the lacI gene for producing a lac repressor protein into the chromosome of the prokaryotic host.

Also provided herein is an expression cassette comprising at least one heterologous gene configured to produce at least one heterologous protein of interest, the gene of interest including

a) one or more coding sequences encoding the one or more proteins of interest,

b) a promoter operably linked to the coding sequence, and

c) at least one lac operator (lacO) operably linked to said promoter.

Specifically, the affinity of LacI to the at least one lacO comprised in the expression cassette is lower than the affinity of LacI to the lac operators lacO1 and lacO3 of the lac operon of a host cell. Preferably, said lac operon is the lac operon endogenous to the host cell.

According to a specific embodiment of the expression cassette provided herein, the heterologous gene configured to produce at least one heterologous protein of interest includes two lac operators, which are at least 92 or 94 bp apart, wherein one lac operator is located within the sequence of the promoter and the second lac operator is upstream of the promoter. Preferably, said two lac operators are at least about 92 to 134 bps apart, preferably they are at least about 103, 105, 114, 116, 125 or 136 or 138 or 149 bps apart. Specifically, said two lac operators are 92, 94, 103, 105, 114, 116, 125, 136, 138 or 149 bps apart.

According to a specific embodiment of the expression cassette provided herein, the heterologous gene configured to produce at least one heterologous protein of interest comprises a lacO1 operator within the sequence of the promoter operably linked to the coding sequence and a native T7A1 initial transcribed sequence (SEQ ID NO:2). Specifically, said expression cassette further comprises a heterologous lacI promoter, which is the lacI^{Q} promoter (SEQ ID NO:1).

Further provided herein is a method of plasmid-free production of a protein of interest in a prokaryotic host on a manufacturing scale, using the expression cassette described herein, comprising the steps of

a) integrating the expression cassette into the chromosome of the prokaryotic host,

b) cultivating the host cells and inducing expression of the gene of interest by addition of an inducer,

c) harvesting protein of interest, and

d) isolating and purifying the protein of interest. and optionally

e) modifying the protein of interest and

f) formulating the protein of interest.

According to a specific embodiment of the method and the system provided herein, the prokaryotic host contains the expression cassette integrated at an attachment site, preferably the attTn7, lacZ, recA, tufa or attnB site.

### FIGURES

FIG. 1: Scheme of integration cartridges. Expression of GFPmut3.1 is controlled by seven different promoter/operator combinations. The T7 expression system is used as reference. The cartridges were cloned into pET30a-cer vector (designated with round brackets) or were integrated into the attTN7 site (designated with squared brackets) of the BL21 genome (B) resp. BL21^{Q} (as described in Example 1) (BQ). In two promoter/operator combinations the wild-type lacI promoter (lacI wt) was exchanged by the lacI^{Q} promoter (lacI^{Q}). LacO1* is a 2 bp truncated version of wild-type lacO1. Sym-lacO is the perfectly symmetric lac operator. +1 T7A1+20 is the native ITS of the T7A1 promoter. Transcription is terminated by tZENIT (tZ). GFPmut3.1 is the coding sequence for expression of the GFPmut3.1 protein. lacO1 is the wild type lacO1. −35 and −10 are the −35 and −10 promoter regions of the respective promoters, A1 and T5.

FIG. 2: Promoter activities of different promoter/operator combinations under uninduced (0 mM IPTG) and induced (0.5 mM IPTG) conditions. The fluorescence of reporter GFPmut3.1 (y-axis) was used to characterize genome-integrated expression systems (A) and plasmid-based expression systems (B). The integration cartridges cloned into pET30a-cer vector are designated with round brackets, those integrated integrated into the attTN7 site of the BL21 genome (B) resp. BL21Q (as described in Example 1) (BQ) are designated with squared brackets.

FIG. 3: Influence of lac-operators on GFP expression and tuneability of expression of GFP expressed by the course of GFP on-line fluorescence (y-axis) in fedbatch-like microtiter cultivation. The dashed vertical lines indicate time of induction. A-D: T5N₂₅ promoter controlled by three lacO (B<3lacO-T5>) (A), two lacO (B<2lacO-T5>) (B), one lacO (B<1lacO-T5>) (C) and one lacO/lacI^{Q} promoter (BQ<1lacO-T5>) (D). E-G: T7_{A1} promoter controlled by two lacO (B<2lacO-A1>) (E), one lacO (B<1lacO-A1>) (F) and one lacO/lacI^{Q} promoter (BQ<1lacO-A1) (G). The T7 expression system is used as reference (H).

FIG. 4: Control of recombinant gene expression with different levels of inducer. Flow cytometry analysis of GFPmut3.1 expression in B<2lacO-A1>, BQ<1lacO-A1> and B3<T7>.

FIG. 5: Scheme of lac-operator binding sites on native lac-operon (top) and gene of interest (bottom). Promoters for the gene of interest are regulated by one lac-operator (A) or two lac-operators that are 62 bp apart (B).

FIG. 6: SEQ ID NOs referred to herein.

FIG. 7: Influence of recombinant expression rate control on LacI concentration. (A) BL21 wild-type cells (lanes 1-3) and B<2lacO-A1> (lanes 4-6) were grown without IPTG (lanes 1 and 4), 0.01 mM IPTG (lanes 2 and 5) and 0.5 mM IPTG (lanes 3 and 6). Proteins of ˜1.2×10⁷ cells were separated by SDS-PAGE and analyzed by western blotting, using an anti-LacI antibody. (B) Fold changes are shown relative to 0 mM IPTG BL21-wt. Error bars indicate standard error of the mean (n=2).

FIG. 8: Process characteristics and product formation kinetic of B3<T7-dFTN2> during the carbon-limited exponential fed-batch cultivation. Cultivations were conducted in a 1.5 L DASGIP® parallel bioreactor system with a final volume of 1.2 L. The dashed vertical lines indicate time of induction.

FIG. 9: Process characteristics and product formation kinetic of BQ<A1-dFTN2> during the carbon-limited exponential fed-batch cultivation. Cultivations were conducted in a 1.5 L DASGIP® parallel bioreactor system with a final volume of 1.2 L. The dashed vertical lines indicate time of induction.

### DETAILED DESCRIPTION

Unless indicated or defined otherwise, all terms used herein have their usual meaning in the art, which will be clear to the skilled person. Reference is for example made to the standard handbooks, such as Sambrook et al, “Molecular Cloning: A Laboratory Manual” (2nd Ed.), Vols. 1-3, Cold Spring Harbor Laboratory Press (1989); Lewin, “Genes IV”, Oxford University Press, New York, (1990), and Janeway et al, “Immunobiology” (5th Ed., or more recent editions), Garland Science, New York, 2001.

The terms “comprise”, “contain”, “have” and “include” as used herein can be used synonymously and shall be understood as an open definition, allowing further members or parts or elements. “Consisting” is considered as a closest definition without further elements of the consisting definition feature. Thus “comprising” is broader and contains the “consisting” definition.

The term “about” as used herein refers to the same value or a value differing by +/−5% of the given value.

Genome integrated, i.e. plasmid-free, expression systems offer significant advantages. Compared to plasmid-based expression systems there is no plasmid mediated metabolic load and no variation in gene dosage during the production process. However, the current state of the art T7-based expression system employing the strong T7 promoter dependent on the T7 RNA polymerase which is under the control of an inducible promoter, still suffers from considerable drawbacks. The strength of the T7 expression system exerts an extreme metabolic load on the host cells. When the gene of interest codes for challenging proteins, the stress and metabolic burden often lead to reduced yield, shortened production periods and even cell death. Moreover, the T7 expression system is leaky, because it shows significant basal expression, and the T7 RNA polymerase is prone to mutations under long-term production conditions.

The plasmid-free inducible expression system provided herein has the profound advantage that the rate of expression is tunable on a single cell level, it exhibits very low basal expression and it is highly efficient in recombinant protein production. Moreover, it provides true control of expression rate, negligible basal expression and a high expression rate even at low inductor concentrations, which is particularly beneficial for production of challenging proteins.

The terms “plasmid-free” or “genome-based” as used herein, refer to an expression system of a protein of interest in a prokaryotic host, wherein the gene for the expression of the protein of interest is located in the genome of the host. Specifically, said gene is an endogenous homologous gene which is located on the chromosome of the prokaryotic host, or is a recombinant heterologous or homologous gene that is integrated into the chromosome of the prokaryotic host.

According to a specific embodiment, a gene for expression of a protein of interest and optionally a lacI gene for expression of a lac repressor protein or a recombinant lacI promoter are integrated into the genome of the host using one or more expression cassette(s) comprising said genes.

Specifically, further recombinant heterologous or homologous genes, such as genes encoding an RNA polymerase or genes encoding helper proteins are introduced into the prokaryotic host. Said further recombinant heterologous or homologous genes may be introduced into the chromosome of the host or may be present in the host cell on a plasmid.

The terms “expression cassette”, or simply “cassette”, synonymously used with “expression cartridge” or simply “cartridge”, refer to a linear or circular DNA construct to be integrated into the prokaryotic genome, such as the bacterial genome. As a result of integration, the expression host cell has an integrated expression cassette. Preferably, the cassette is a linear DNA construct comprising essentially a promoter, a gene of interest, immediately upstream of the gene of interest a Shine-Dalgarno (SD) sequence, also termed ribosome binding site (RBS) and two terminally flanking regions which are homologous to a genomic region and which enable homologous recombination. In addition, the cassette may contain other sequences such as for example sequences coding for antibiotic selection markers, prototrophic selection markers or fluorescent markers, markers coding for a metabolic gene, genes which improve protein expression or two flippase recognition target sites (FRT) which enable the removal of certain sequences (e.g. antibiotic resistance genes) after integration.

The expression cassette is synthesized and amplified by methods known in the art, in the case of linear cassettes, usually by standard polymerase chain reaction, PCR. Since linear cassettes are usually easier to construct, they are preferred for obtaining the expression host cells used in the system and method provided herein. Moreover, the use of a linear expression cassette provides the advantage that the genomic integration site can be freely chosen by the respective design of the flanking homologous regions of the cassette. Thereby, integration of the linear expression cassette allows for greater variability with regard to the genomic region.

Expression vectors comprise the expression cassette described herein and in addition optionally comprises flanking regions homologous to the genome integration site, a number of restriction enzyme cleavage sites, an initial transcribed sequence (ITS) and a transcription terminator, and optionally one or more selectable markers (e.g., an amino acid synthesis gene or a gene conferring resistance to antibiotics such as ampicillin, kanamycin, chloramphenicol or streptomycin), which components are operably linked together. A common type of vector is a “plasmid”, which generally is a self-contained molecule of double-stranded DNA that can readily accept additional (foreign) DNA and which can readily be introduced into a suitable host cell. Specifically, the term “vector” or “plasmid” refers to a vehicle by which a DNA or RNA sequence (e.g. a foreign gene) can be introduced into a host cell, so as to transform the host and promote expression (e.g. transcription and translation) of the introduced sequence.

As used herein, the term “prokaryotic host” refers to any bacterial host, in particular it refers to bacterial host cells. In principle, there are no limitations regarding the choice of bacterial host cells, except for certain specific requirements detailed below. The bacterial host cells may be eubacteria (gram-positive or gram-negative) or archaebacteria, as long as they allow genetic manipulation for insertion of a gene of interest, advantageously for site-specific integration. Preferably, the bacterial host cells allow cultivation on a manufacturing scale. Preferably, the host cell has the property to allow cultivation to high cell densities. Examples for bacterial host cells that have been shown to be suitable for recombinant industrial protein production are Escherichia coli, Bacillus subtilis, Pseudomonas fluorescens as well as variations thereof and Lactococcus lactis strains. Preferably, the host cells are E. coli cells.

A requirement to the host cell is that it comprises an RNA polymerase that can bind to the promoter controlling the gene encoding the protein of interest.

In certain embodiments, the host cell carries, in its genome, a marker gene in view of selection.

In view of site-specific gene insertion, another requirement to the host cell is that it contains at least one genomic region (either a coding or any non-coding functional or non-functional region or a region with unknown function) that is known by its sequence and that can be disrupted or otherwise manipulated to allow insertion of a heterologous sequence, without being detrimental to the cell.

With regard to the integration locus, the expression system used in the invention allows for a wide variability. In principle, any locus with known sequence may be chosen, with the proviso that the function of the sequence is either dispensable or, if essential, can be complemented (as e.g. in the case of an auxotrophy).

Integration of the gene of interest into the bacterial genome can be achieved by conventional methods, e.g. by using linear cartridges that contain flanking sequences homologous to a specific site on the chromosome, as described for the attTn7-site, e.g. in (30). Moreover, the use of a linear expression cartridge provides the advantage that the genomic integration site can be freely chosen by the respective design of the flanking homologous regions of the cartridge. Thereby, integration of the linear expression cartridge allows for greater variability with regard to the genomic region. In a preferred embodiment, integration of a linear cartridge is at an attachment site like the attB site or the attTn7 site, which are well-proven integration sites. Examples, without limitation, of other integration methods useful in the present invention are e.g. those based on Red/ET recombination, e.g. described in (31). Alternatively, an expression cassette can first be integrated into the genome of an intermediate donor host cell, from which it can then be transferred to the host cell by transduction by the P1 phage, e.g. described in (32). The integration method used herein is not limited to the above-mentioned examples; rather any integration method known in the art can be used.

The integration methods for obtaining the expression host cell are not limited to integration of one gene of interest at one site in the genome; they allow for variability with regard to both the integration site and the expression cassettes. By way of example, more than one gene of interest may be inserted, i.e. two or more identical or different sequences under the control of identical or different promoters can be integrated into one or more different loci on the genome. By way of example, it allows expression of two different proteins that form a heterodimeric complex. Heterodimeric proteins consist of two individually expressed protein Subunits, e.g. the heavy and the light chain of a monoclonal antibody or an antibody fragment.

Although the invention allows plasmid-free production of a protein of interest, it does not exclude that in the expression host cell a plasmid may be present that carries sequences to be expressed other than the gene of interest, e.g. helper proteins and/or recombination proteins. Preferably, care should be taken that in such embodiments the advantages of the invention should not be overruled by the presence of the plasmid, i.e. the plasmid should be present at a low copy number and should not exert a metabolic burden onto the cell.

Integration of one or more recombinant genes into the genome results in a discrete and pre-defined number of genes of interest per cell. In the embodiment of the invention that inserts one copy of the gene, this number is usually one (except in the case that a cell contains more than one chromosome or genome, as it occurs transiently during cell division), as compared to plasmid-based expression which is accompanied by copy numbers up to several hundred. In the expression system used in the method of the present invention, by relieving the host metabolism from plasmid replication, an increased fraction of the cells synthesis capacity is utilized for recombinant protein production.

A particular advantage is that the inducible expression system described herein has no limitations with regard to the level of induction. This means that the system cannot be “over-induced as it often occurs in plasmid-based systems, or systems employing strong promoters such as the T7 expression system. Since the genome-based expression system allows exact control of protein expression, it is particularly advantageous in combination with expression targeting pathways that depend or rely on well-controlled expression. In a preferred embodiment, the method of the invention includes secretion (excretion) of the protein of interest from the bacterial cytoplasm into the periplasm and/or culture medium. The advantage of this embodiment is an optimized and sustained protein secretion rate, resulting in a higher titer of secreted protein as compared to prior art secretion systems. Specifically, this can be achieved by fusing a signal peptide N-terminal to the protein of interest/a nucleotide sequence encoding a signal peptide, which leads the protein of interest to the transporters of the host, causes translocation into the periplasma of the host and is cleaved by the signal peptidase of the host. Any signal peptide known in the art can be used such as but not limited to the ompA-, pelB, malE-, phoA-, dsbA-, lysC-, lolB-, pyrL-leader peptides.

As used herein, the term “RNA polymerase (RNAP) gene” refers to a gene expressing an RNAP, which gene is comprised in the genome, e.g. in a plasmid, or chromosome of the prokaryotic host. Preferably, said gene expresses an RNAP that is endogenous to the prokaryotic host.

In bacteria, the same enzyme catalyzes the synthesis of mRNA and non-coding RNA (ncRNA). RNAP is a large molecule; the core enzyme has five subunits (˜400 kDa). In order to bind promoters, RNAP core associates with the transcription initiation factor sigma (a) to form RNA polymerase holoenzyme. Sigma reduces the affinity of RNAP for nonspecific DNA while increasing specificity for promoters, allowing transcription to initiate at correct sites. The complete holoenzyme therefore has 6 subunits (˜450 kDa). The core enzyme is responsible for binding to template DNA to synthesize RNA, which is complemented by a σ factor to form a holoenzyme that recognizes the promoter sequence to begin promoter-specific transcription.

According to a preferred embodiment, the prokaryotic host cells of the system described herein are E. coli cells and the RNAP is an RNAP that is endogenous to E. coli, most preferably it is σ⁷⁰ E. coli RNA polymerase. The σ subunit of bacterial RNA polymerase (RNAP) is required for promoter-specific transcription initiation. In the case of E. coli and other gram-negative rod-shaped bacteria, the “housekeeping” or “primary” sigma factor is σ⁷⁰. Every cell has a “housekeeping” sigma factor that keeps essential genes and pathways operating. When complexed with the RNAP core enzyme (subunit structure α₂ββ′ω), different σ factors specify the recognition of different classes of promoters. Genes recognized by σ⁷⁰ all contain similar promoter consensus sequences consisting of two parts. The primary σ factor in Escherichia coli, σ⁷⁰, typically directs transcription initiation from promoters defined by two conserved hexameric DNA sequence elements, termed the −10 and −35 elements for their relationship to the transcription start site (position +1). Relative to the DNA base corresponding to the start of the RNA transcript, the consensus promoter sequences are characteristically centered at 10 and 35 nucleotides before the start of transcription (−10 and −35).

The term “expression” is understood in the following way. Nucleic acid molecules containing a desired coding sequence of an expression product such as e.g., a recombinant protein as described herein, and control sequences such as e.g., a promoter in operable linkage, may be used for expression purposes. Hosts transformed or transfected with these sequences are capable of producing the encoded proteins. In order to effect transformation, the expression system may be included in a vector; however, most preferably the relevant DNA is integrated into the host chromosome.

The term “gene” as used herein refers to a DNA sequence that comprises at least promoter DNA, optionally including operator DNA, and coding DNA which encodes a particular amino acid sequence for a particular polypeptide or protein. Promoter DNA is a DNA sequence which initiates, regulates, or otherwise mediates or controls the expression of the coding DNA. Promoter DNA and coding DNA may be from the same gene or from different genes, and may be from the same or different organisms.

The term “recombinant” as used herein shall mean “being prepared by or the result of genetic engineering”. A recombinant host specifically comprises a recombinant expression vector or cloning vector, or it has been genetically engineered to contain a recombinant nucleic acid sequence, in particular employing nucleotide sequence foreign to the host. A recombinant protein is produced by expressing a respective recombinant nucleic acid in a host.

With regard to the protein of interest (POI), there are no limitations. More specifically, the protein may either be a polypeptide not naturally occurring in the host cell, i.e. a heterologous protein, or else may be native to the host cell, i.e. a homologous protein to the host cell, but is produced, for example, upon integration by recombinant techniques of one or more copies of the nucleic acid sequence encoding the homologous POI into the genome or chromosome of the host cell, or by recombinant modification of the promoter sequence controlling the expression of the gene encoding the POI. The POI can be a monomer, dimer or multimer, it can be a homomer or heteromer.

Examples for proteins that can be produced by the method of the invention are, without limitation, enzymes, regulatory proteins, receptors, peptides, e.g. peptide hormones, cytokines, membrane or transport proteins. The proteins of interest may also be antigens as used for vaccination, vaccines, antigen-binding proteins, immune stimulatory proteins, allergens, full-length antibodies or antibody fragments or derivatives. Antibody derivatives may be for example single chain variable fragments (scFv), Fab fragments or single domain antibodies.

The DNA molecule encoding the protein of interest is also termed “gene of interest”. Specifically, the gene of interest includes the DNA sequence encoding the protein of interest, a promoter operably linked to the coding sequence and at least one lac operator within the sequence of the promoter.

Further, the gene of interest encoding the POI can be a naturally existing DNA sequence or a non-natural DNA sequence. One or more gene of interests can be under the control of one promoter as described herein. Alternatively, each gene of interest is under one promoter. The gene of interests may all be on the same expression cassette or on multiple expression cassettes. The POI can be modified in any way. Non-limiting examples for modifications can be insertion or deletion of post-translational modification sites, insertion or deletion of targeting signals (e.g.: leader peptides), fusion to tags, proteins or protein fragments facilitating purification or detection, mutations affecting changes in stability or changes in solubility or any other modification known in the art. In certain embodiments of the invention the recombinant protein is a biopharmaceutical product, which can be any protein suitable for therapeutic or prophylactic purposes in mammals.

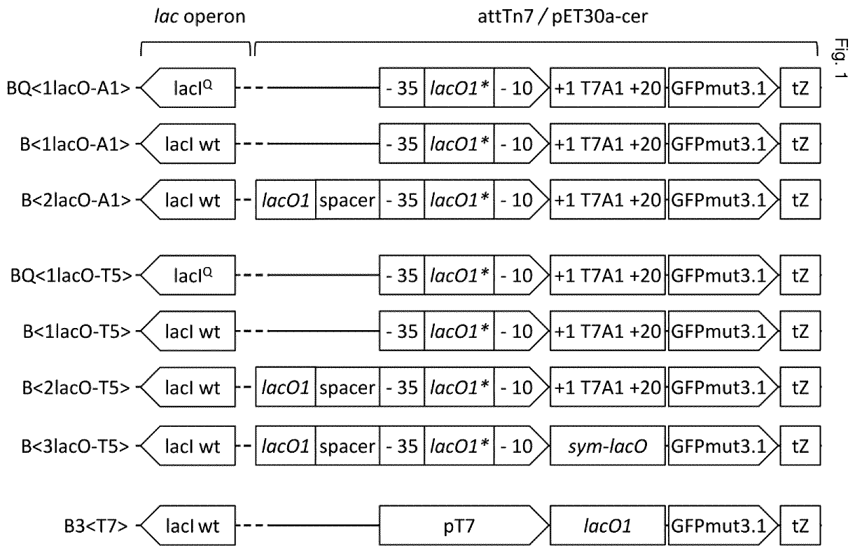

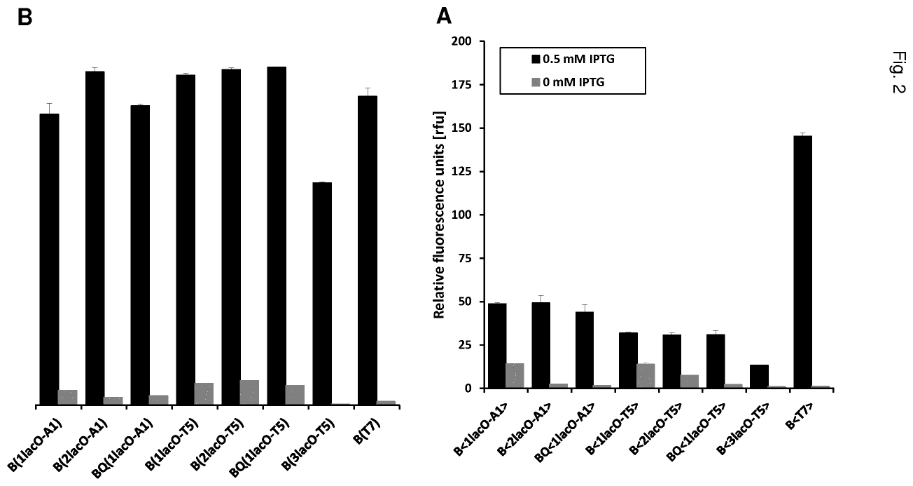

The term “promoter” as used herein refers to an expression control element that permits binding of RNA polymerase and the initiation of transcription. Specifically, the promoter operably linked to the gene of interest as described herein, comprises at least one lac operator within its sequence. Specifically, said at least one lac operator is situated between the −10 and −35 elements, which elements are preferably located 10 and 35 nucleotides before the start of transcription (−10 and −35), as exemplified in FIG. 1.

The lac promoter is the promoter of the lac operon, which controls transcription of the three lac genes, IacZ, lacY and lacA. The wildtype lac promoter does not comprise a lac operator within its sequence, as it does not comprise a lacO between the −10 and −35 promoter elements. Preferably, in the inducible expression system described herein, the lac promoter is the endogenous lac promoter comprising the endogenous lac operators. According to a specific embodiment, one or more lac operators of the endogenous lac promoter are genetically modified to increase their binding affinity to the lac repressor molecule LacI. Specifically, they are genetically modified so that their affinity to the lac repressor molecule LacI is greater than the affinity of the lac operators of the promoter operably linked to the gene of interest.

The lacI promoter as used herein, is the promoter operably linked to the coding sequence of the lacI gene. Specifically, the inducible system described herein, includes the wild-type lacI promoter or a genetically modified lacI promoter which increases expression of LacI, such as the exemplary lacI^{Q} promoter described herein. Specifically, the lacI promoter is a constitutive promoter. Specifically, any constitutive promoter stronger than the native lacI promoter can be used as lacI promoter according to the present invention. Specifically, any promoter stronger than the native lacI promoter can be used as lacI promoter according to the system provided herein, such as but not limited to T5, T7A1, T7A2, T7A3, T7, dnaK/J, spac, bla, nptII, cat promoters.

The promoter operably linked to the gene encoding the protein of interest as described herein, can be any inducible promoter that is recognized by an RNAP encoded by an RNAP gene comprised in the chromosome of the host.

According to certain embodiments of the invention, the gene of interest may be under the control of the lac, lacUV5, tac or the trc promoter, the lac or the lacUV5 promoter, the T5 promoters (Gentz and Bujard, 1985), such as the T5_{N25}, or the T7 promoters (Hawley and McClure, 1983), such as T7 C or T7 D or the T7A promoters, such as T7A1, T7A2 or T7A3 promoters (all inducible by lactose or its analogue IPTG), or other promoters suitable for recombinant protein expression, which all use E. coli RNA polymerase. The sequences of such promoters are well known in the art, such as e.g. those described by Gentz and Bujard, 1985 (33) or Hawley and McClure, 1983 (38). Specifically, the sequences of said promoters are modified to comprise at least one lacO within their sequence, as described herein.

According to a specific embodiment, the promoter described herein, which is in operable linkage to the sequence encoding the protein of interest, comprises a lacO within its sequence. In bacteria, the sequence of a promoter typically contains two short sequence elements, which, in wild type promoters, are typically approximately 10 and 35 nucleotides upstream of the transcription start site. These sequences are conserved among many bacterial strains. For example, the sequence at −10 nucleotides (also called the −10 element) typically has the consensus sequence TATAAT (SEQ ID NO:34), and the sequence at −35 (also called the −35 element) has the consensus sequence TTGACA (SEQ ID NO:35). The above consensus sequences, while conserved on average, are not found intact in all promoters. On average, only 3 to 4 of the 6 base pairs in each consensus sequence are found in any given promoter. Few natural promoters have been identified to date that possess intact consensus sequences at both the −10 and −35 elements. Specifically, artificial promoters with complete conservation of the −10 and −35 elements transcribe at lower frequencies than those with a few mismatches with the consensus.

Specifically, the promoter described herein comprises at least one lacO between the −10 and −35 elements.

The term “inducer”, synonymously used with “inductor”, refers the factor capable of leading to the induction of transcription through direct or indirect regulation of promoter activity. Specifically, as used herein, inducer is any factor that is capable of binding the lac repressor molecule and inhibiting its interaction with the promoter operably linked to the gene of interest. Preferably, the inducer used herein is isopropylthiogalactoside (IPTG), lactose, methyl-β-D-thiogalactoside, phenyl-β-D-galactose or ortho-nitrophenyl-β-galactoside (ONPG).

There is no limitation as regards the mode by which induction of protein expression is performed. By way of example, the inductor can be added as a singular or multiple bolus or by continuous feeding, the latter being also known as “inductor feed(ing)”. There are no limitations as regards the time point at which the induction takes place. The inductor may be added at the beginning of the cultivation or at the point of starting continuous nutrient feeding or after (beyond) the start of feeding. Inductor feeding may be accomplished by either having the inductor contained in the culture medium or by separately feeding it. The advantage of inductor feeding is that it allows to control inductor dosage, i.e. it allows to maintain the dosage of a defined or constant amount of inductor per constant number of genes of interest in the production system. For instance, inductor feeding allows an inductor dosage which is proportional to the biomass, resulting in a constant ratio of inductor to biomass. Biomass units on which the inductor dosage can be based, may be for instance cell dry weight (CDW), wet cell weight (WCW), optical density, total cell number (TCN; cells per volume) or colony forming units (CFU per volume) or on-line monitored signals which are proportional to the biomass (e.g. fluorescence, turbidity, light scatter, dielectric capacity, carbon dioxide concentration in the exhaust gas etc.). Essentially, the method of the invention allows the precise dosage of inductor per any parameter or signal which is proportional to biomass, irrespective of whether the signal is measured off-line or online. Since the number of genes of interest is defined and constant per biomass unit (one or more genes per cell), the consequence of this induction mode is a constant dosage of inductor per gene of interest. As a further advantage, the exact and optimum dosage of the amount of inductor relative to the amount of biomass can be experimentally determined and optimized.

It may not be necessary to determine the actual biomass level by analytical methods. For instance, it may be sufficient to add the inductor in an amount that is based on previous cultivations (historical biomass data). In another embodiment, it may be preferable to add the amount of inductor per one biomass unit as theoretically calculated or predicted. For instance, it is well known for feeding-based cultivations (like fed-batch or continuous) that one unit of the growth-limiting component in the feed medium, usually the carbon source, will result in a certain amount of biomass.

Preferably, the inducer is used at a concentration ranging from 0.005 mM to 1 mM, even more preferably from 0.01 mM to 0.5 mM. Specifically, the concentration of IPTG is in the range of 1-100 μmol/g CDW.

As provided herein, the host used in the inducible expression system described herein comprises a lac operon, preferably a wild-type lac operon, and a lacI gene.

As referred to herein, the endogenous lac operon contains three genes: IacZ, lacY, and lacA. These genes are transcribed as a single mRNA, under control of one promoter. In addition to the three genes, the lac operon comprises the lac promoter and the lac operators lacO1, lacO2 and lacO3. The lac promoter is the binding site for the RNA polymerase. The lac operator is the negative regulatory site bound by the lac repressor protein. The operator overlaps with the promoter, and when the lac repressor protein is bound, RNA polymerase cannot bind to the promoter and start transcription. According to a specific embodiment, the endogenous lac operon is modified to increase the binding affinity of LacI to at least one of the lac operators lacO1, lacO2 or lacO3. Specifically, at least one of the lac operators lacO1, lacO2 or lacO3 is modified, i.e. the endogenous lac operon comprises a functional variant of lacO1, lacO2 and/or lacO3 with increased affinity for LacI.

As used herein, the term “lad gene” refers to a gene for expression of the lac repressor protein, also called lac inhibitor (LacI), or any functional variant thereof with at least 30% sequence identity to lacI (SEQ ID NO:26). Specifically, said gene comprises a lacI coding sequence, a lacI promoter operably linked to the lacI coding sequence, wherein the lacI promoter is selected from the group consisting of the wild-type lacI promoter and a lacI promoter which increases expression of lacI. Specifically, the lacI gene expresses LacI or a functionally active variant thereof comprising at least 40, 50, 60, 70, 80 or 90% sequence identity to LacI (SEQ ID NO:27). Specifically, the lacI promoter which increases expression of LacI is a strong promoter, which increases expression of LacI by at least 1.5, 2, 2.5 or 5-fold, preferably 10-fold or more. Specifically, it increases the expression of LacI by at least 20-fold, 30-fold, 40-fold, 50-fold, 60-fold, 70-fold, 80-fold, 90-fold or even 100-fold. An exemplary embodiment of the inducible system provided herein comprises the lacI^{Q} promoter as the lacI promoter which increases expression of lacI. The lacI^{Q} promoter includes a point mutation, a single C→T change, in the promoter region upstream of the native lacI gene, resulting in a 10-fold increase in mRNA transcription. The promoter for the lacI coding sequence may include the native lacI initiation codon or any variants thereof. The lacI gene is preferably incorporated into the host's chromosomal DNA or contained on a single-copy vector.

In wild-type E. coli, the lac repressor protein forms a homo-tetramer that binds to the lac-operator sequences (lacO) and represses the transcription of the lacZYA operon. In the presence of lactose or the non-metabolizable isopropyl β-D-1-thiogalactopyranoside (IPTG), LacI changes its structure and can no longer bind to the lac-operator, resulting in induction of transcription. The lac-operator sites are DNA sequences with inverted repeat symmetry.

The higher the symmetry, the greater the binding affinity of LacI to the operator sequence. An artificial perfectly symmetric lacO (sym-lacO) was found to bind LacI with the greatest affinity, whereas the three wild-type operators lacO1, lacO2 and lacO3 exhibiting an approximate symmetry showed lower affinities, resulting in the following order with respect to the affinity to LacI: sym-lacO>lacO1>lacO2>lacO3. LacI binds simultaneously to both, the primary operator lacO1 and to either lacO2 or lacO3 through a DNA-looping mechanism. LacO2 is located 401 bp downstream of lacO1, whereas lacO3 lies only 92 bp upstream of lacO1. The main contribution to repression comes from the DNA-looping of lacO1 and lacO3 due to their closer proximity. Furthermore, when lacO1 and lacO3 are bound by LacI, the production of LacI itself is prevented. The 3′ end of the lacI gene overlaps with lacO3. In a repressed state, transcription of lacI results in a truncated mRNA, which is rapidly degraded by the cell. Due to this autoregulation, the concentration of the LacI tetramer is ˜40 molecules in induced cells and ˜15 molecules in non-induced cells.

Sequences of lac operators are well known in the art. Exemplary lac operator sequences are provided by SEQ ID NO:3-5.

Suitable variants of the nucleic acid or polypeptide sequences, specifically lacO1, lacO2 and lacO3, disclosed herein are functional variants having the same type of activity (without regard to the degree of the activity) as the nucleic acid or polypeptide to which the sequence corresponds. Such activities may be tested according to the assays described in the Examples below and according to methods known in the art.

The term “functional variant” or functionally active variant also includes naturally occurring allelic variants, as well as mutants or any other non-naturally occurring variants. As is known in the art, an allelic variant is an alternate form of a nucleic acid or peptide that is characterized as having a substitution, deletion, or addition of one or nucleotides or more amino acids that does essentially not alter the biological function of the nucleic acid or polypeptide.

Functional variants may be obtained by sequence alterations in the polypeptide or the nucleotide sequence, e.g. by one or more point mutations, wherein the sequence alterations retains or improves a function of the unaltered polypeptide or the nucleotide sequence, when used in combination of the invention. Such sequence alterations can include, but are not limited to, (conservative) substitutions, additions, deletions, mutations and insertions.

A point mutation is particularly understood as the engineering of a poly-nucleotide that results in the expression of an amino acid sequence that differs from the non-engineered amino acid sequence in the substitution or exchange, deletion or 5 insertion of one or more single (non-consecutive) or doublets of amino acids for different amino acids.

An exemplary functional variant of the lacO1 operator is a 2 base-pair truncated version of wild-type lacO1, which comprises a deletion of 2 bp at its 5′ end, lacO* (SEQ ID NO:6).

Transcription rate control, also referred to as fine-tuning of protein production or “tunability” is highly relevant in bioprocessing. Bioprocesses are designed to maximally exploit the cells' synthesizing capacity during a maximal long period, yielding properly folded and processed protein. But, strong expression systems, such as e.g. the T7 expression system, are known to exhibit an “all-or-none” behavior, where the reduced expression level in partially induced cultures is the result of the formation of subpopulations of fully induced and non-induced cells. Such problem is solved by the inducible expression system described herein which allows tunability, specifically single-cell tunability. In the inducible expression system described herein, the affinity of LacI to the at least one lacO of the promoter operably linked to the gene of interest is lower than the affinity of LacI to the lac operators lacO1 and lacO3 of the endogenous lac operon of the host. If the binding constant (Kₐ) of LacI to the at least one lacO at the gene of interest (GOI) is higher than the binding constant to the lacO at the lac-operon, the first LacI molecules, which are not inactivated by IPTG will preferentially bind to the lacO binding sites of the GOI instead of the lacO3/lacO1 on the lac-operon. Hence, autoregulation of LacI does not intervene and more LacI molecules are being produced leading to an overregulation of the system which results in a complete stop of transcription of the gene of interest in this cell. In particular, at low inducer concentrations, such a system leads to at least two distinct sub-populations, of POI producing and non-producing cells, as such expression systems stop their productivity, but still continue to grow.

In the inducible expression system described herein, however, the binding constant (Kₐ) of LacI to the at least one lacO at the gene of interest (GOI) is lower than the binding constant to the lacO at the lac-operon. Therefore, LacI preferentially binds to the operators of the endogenous lac operon, preventing transcription of the three lacZ, lacY and lacA genes and also preventing further production of LacI through the autoregulation of LacI, resulting in a homogenous population at any given inducer concentration.

As used herein, the term “affinity” or “binding affinity” refers to strength of association between a ligand and a receptor as defined by the dissociation and/or the association constant. Dissociation constant (K_{d}) is the rate constant of dissociation at equilibrium, defined as the ratio k_{off}/kₒₙ, wherein k_{off} is the rate constant of dissociation of the ligand from the receptor and kₒₙ is the rate constant of association of the ligand to the receptor. The Association constant (Kₐ) is the opposite of K_{d}. When Kₐ is high, K_{d} is low, and the ligand has a high affinity for the receptor (fewer molecules are required to bind 50% of the receptors).

Usually a binder is considered a high affinity binder with a dissociation constant of at least K_{d}<10⁻⁷ M, in some cases higher affinities are required such as, e.g. K_{d}<10⁻⁸ M, preferably K_{d}<10⁻⁹ M, even more preferred is K_{d}<10⁻¹° M.

In the inducible expression system described herein, the binding affinity of LacI to the one or more lacO/lacOs of the gene of interest is lower than the affinity of LacI to the lac operators lacO1 and lacO3 of the endogenous lac operon. Specifically, lacI binds to the lac operators lacO1 and lacO3 with a K_{d} of at least K_{d}<10⁻⁷ M, preferably K_{d}<10⁻⁸ M, preferably K_{d}<10⁻⁹ M, even more preferred is K_{d}<10⁻¹⁰ M. Specifically, LacI binds to the one or more lacO/lacOs of the gene of interest with a K_{d} that is increased by at least 5, 10, 15, 20, 30, 40, 50, 60, 70, 80, 90 or 100% or more. Consequently, LacI binds to the one or more lacO/lacOs of the gene of interest with a Kₐ that is about 5, 10, 15, 20, 30, 40, 50, 60, 70, 80 or 90% lower than the Kₐ of LacI to the lacO1 and lacO3 of the endogenous lac operon.

Specifically, binding affinity is determined by an affinity ELISA assay. In certain embodiments binding affinity is determined by a BIAcore, ForteBio or MSD assay. In certain embodiments binding affinity is determined by a kinetic method. In certain embodiments binding affinity is determined by an equilibrium/solution method. Those skilled in the art can determine appropriate parameters to determine binding affinity of a ligand to a certain molecule. The binding affinity can be routinely determined by one skilled in the art.

“Sequence identity” or “percent (%) amino acid sequence identity” as described herein is defined as the percentage of nucleotides or amino acid residues in a candidate sequence that are identical with the nucleotides or amino acid residues in the specific nucleotide or polypeptide sequence to be compared (the “parent sequence”), after aligning the sequence and introducing gaps, if necessary, to achieve the maximum percent sequence identity, and not considering any conservative substitutions as part of the sequence identity. Those skilled in the art can determine appropriate parameters for measuring alignment, including any algorithms needed to achieve maximal alignment over the full length of the sequences being compared.

The term “operably linked” as used herein refers to the association of nucleotide sequences on a single nucleic acid molecule, i.e. the vector, in a way such that the function of one or more nucleotide sequences is affected by at least one other nucleotide sequence present on said nucleic acid molecule. For example, a promoter is operably linked with a coding sequence encoding the protein of interest, when it is capable of effecting the expression of that coding sequence. Specifically, such nucleic acids operably linked to each other may be immediately linked, i.e. without further elements or nucleic acid sequences in between or may be indirectly linked with spacer sequences or other sequences in between. Specifically, in the context of a lac operator being operably linked to a promoter refers to the ability of the lac operator to regulate the ability of the promoter to control expression of the coding sequence under specific conditions. Such as the ability of the lac operator to inhibit promoter-dependent expression of the gene of interest when lac repressor protein is bound thereto.

The term “heterologous” as used herein with respect to a nucleotide or amino acid sequence or protein, refers to a compound which is either foreign, i.e. “exogenous”, such as not found in nature, to a given host cell; or that is naturally found in a given host cell, e.g., is “endogenous”, however, in the context of a heterologous construct, e.g., employing a heterologous nucleic acid, thus “not naturally-occurring”. The heterologous nucleotide sequence as found endogenously may also be produced in an unnatural, e.g., greater than expected or greater than naturally found, amount in the cell. The heterologous nucleotide sequence, or a nucleic acid comprising the heterologous nucleotide sequence, possibly differs in sequence from the endogenous nucleotide sequence but encodes the same protein as found endogenously. Specifically, heterologous nucleotide sequences are those not found in the same relationship to a host cell in nature (i.e., “not natively associated”). Any recombinant or artificial nucleotide sequence is understood to be heterologous. An example of a heterologous polynucleotide or nucleic acid molecule comprises a nucleotide sequence not natively associated with a promoter, e.g., to obtain a hybrid promoter, or operably linked to a coding sequence, as described herein. As a result, a hybrid or chimeric polynucleotide may be obtained. A further example of a heterologous compound is a P01 encoding polynucleotide or gene operably linked to a transcriptional control element, e.g., a promoter, to which an endogenous, naturally-occurring P01 coding sequence is not normally operably linked.

The invention furthermore comprises the following items:

1. A genome-based expression system for production of a protein of interest (POI) in a prokaryotic host, comprising at least

a) an RNA polymerase (RNAP) gene,

b) a gene encoding a POI, comprising
a coding sequence,
a promoter operably linked to said coding sequence, wherein said promoter is recognized by the RNAP expressed from a), and
at least one lac operator (lacO) within the sequence of said promoter; and

c) a lacI gene for expression of a lac repressor protein (LacI) comprising
a lacI coding sequence,
a lacI promoter operably linked to the lacI coding sequence, wherein the lacI promoter is a wild-type lacI promoter or a lacI promoter which increases LacI expression;

wherein the expression rate of the protein of interest is regulated by an inducer binding LacI.

2. The genome-based expression system of item 1, wherein the gene encoding a POI contains (i) one lacO within the sequence of the promoter or (ii) one lacO within the sequence of the promoter and one lacO upstream of the first lacO.

3. The genome-based expression system of item 1 or 2, wherein the gene encoding a POI contains one lacO within the sequence of the promoter, and the lacI promoter is a promoter which increases LacI expression.

4. The genome-based expression system of any one of items 1 to 3, wherein the gene encoding a POI contains one lacO within the sequence of the promoter and one lacO upstream of the first lacO, and the lacI promoter is a promoter which increases LacI expression.

5. The genome-based expression system of any one of items 1 to 4, wherein the prokaryotic host is Escherichia coli (E. coli).

6. The genome-based expression system of any one of items 1 to 5, wherein the host is E. coli of the strain BL21 or K-12.

7. The genome-based expression system of any one of items 1 to 6, wherein the RNAP is a heterologous or homologous RNAP, preferably the RNAP is an RNAP homologous to the host, specifically it is an E. coli RNA polymerase, preferably the σ⁷⁰ E. coli RNA polymerase.

8. The genome-based expression system of any one of items 1 to 7, wherein the promoter in b) of item 1 is selected from the group consisting of T5, T5_{N25}, T7A1, T7A2, T7A3, lac, lacUV5, tac or trc.

9. The genome-based expression system of any one of items 1 to 8, wherein the lacI promoter is the lacI promoter which increases LacI expression, which is the lacI^{Q} promoter (SEQ ID NO:1).

10. The genome-based expression system of any one of items 1 to 9, wherein the lac operator is a lacO1 (SEQ ID NO:3), lacO2 (SEQ ID NO:4) or lacO3 (SEQ ID NO:5).

11. The genome-based expression system of item 10, wherein the lac operator is a functional variant of lacO1, lacO2 or lacO3 with at least 65% sequence identity or a perfectly symmetric lacO.

12. The genome-based expression system of any one of items 1 to 11, wherein said promoter operably linked to the coding sequence encoding the protein of interest comprises an initial transcribed sequence (ITS), preferably a native T7A1 initial transcribed sequence (SEQ ID NO:2).

13. The genome-based expression system of any one of items 1 to 12, wherein the inducer is selected from the group consisting of isopropylthiogalactoside (IPTG), lactose, methyl-β-D-thiogalactoside, phenyl-β-D-galactose and ortho-Nitrophenyl-β-galactoside (ONPG).

14. The genome-based expression system of any one of items 1 to 13, wherein the gene for expression of a protein of interest contains one lacO1 operator within the sequence of the promoter operably linked to the coding sequence and the native T7A1 initial transcribed sequence (SEQ ID NO:2), and wherein the lacI promoter is a lacI^{Q} promoter.

15. The genome-based expression system of any one of items 1 to 14, wherein the gene of interest contains two lac operators which are at least 92 or 94 basepairs (bps) apart, preferably 103, 105, 114, 116, 125, 127, 134, 136, 138 or 149 bps apart, wherein one lac operator is located within the sequence of the promoter operably linked to the coding sequence and the second lac operator is upstream of the promoter.

16. The genome-based expression system of any one of items 1 to 15, wherein the gene encoding the protein of interest is a heterologous gene.

17. The system of any one of items 1 to 16, wherein at least one lac operator of the lac operon of the prokaryotic host is genetically modified to increase its binding affinity to the lac repressor molecule LacI.

18. A method of plasmid-free production of a protein of interest in a prokaryotic host, using the genome-based expression system of any one of items 1 to 17, comprising the steps of

a) inducing expression of the gene encoding the POI by addition of an inducer,

b) harvesting the POI,

c) isolating and purifying the POI, and optionally

d) modifying, and

e) formulating the POI.

19. An expression cassette comprising at least one heterologous gene configured to produce at least one heterologous POI, including

a) one or more coding sequences encoding the one or more POI,

b) a promoter operably linked to the one or more coding sequences, and

c) at least one lac operator (lacO) within the sequence of said promoter;

wherein the affinity of LacI to lacO of c) is lower than the affinity of LacI to the lac operators lacO1 and lacO3 of the endogenous lac operon of a host cell.

20. The expression cassette of item 19, wherein the heterologous gene configured to produce at least one heterologous protein of interest includes two lac operators, which are at least 92 or 94 bp apart, wherein one lac operator is located within the sequence of the promoter and the second lac operator is upstream of the promoter.

21. The expression cassette of item 19 or 20, further comprising a heterologous lacI promoter, which is the lacI^{Q} promoter (SEQ ID NO:1).

22. The expression cassette of any one of items 19 to 21, wherein the heterologous gene configured to produce at least one heterologous POI comprises a lacO1 operator within the sequence of the promoter operably linked to the coding sequence and a native T7A1 initial transcribed sequence (SEQ ID NO:2).

23. A method of plasmid-free production of a protein of interest in a prokaryotic host on a manufacturing scale, using the expression cassette of any one of items 19 to 22, comprising the steps of

a. integrating the expression cassette into the chromosome of the prokaryotic host,

b. inducing expression of the gene encoding the POI by addition of an inducer,

c. harvesting the POI,

d. isolating and purifying the POI, and optionally

e. modifying, and

f. formulating the POI.

24. An inducible system for plasmid-free production of a protein of interest (POI) in a prokaryotic host, comprising at least

a) an RNA polymerase (RNAP) gene in the chromosome of the host,

b) a gene encoding a POI comprising
a coding sequence,
a promoter operably linked to said coding sequence, wherein said promoter is recognized by the RNAP expressed from a), and
at least one lac operator (lacO) within the sequence of said promoter; and

c) a lacI gene encoding a lac repressor protein (LacI) comprising
a lacI coding sequence,
a lacI promoter operably linked to the lacI coding sequence, wherein the lacI promoter is a wild-type lacI promoter or a lacI promoter which increases LacI expression;

wherein the affinity of LacI to the one or more lacO/lacOs of b) is lower than the affinity of lacI to the lac operators lacO1 and lacO3 of the endogenous lac operon of the host and wherein the expression rate of the POI is regulated by an inducer binding LacI.

25. The system of item 24, wherein at least one lac operator of the lac operon of the prokaryotic host is genetically modified to increase its binding affinity to the lac repressor molecule LacI.

The examples described herein are illustrative of the present invention and are not intended to be limitations thereon. Different embodiments of the present invention have been described according to the present invention. Many modifications and variations may be made to the techniques described and illustrated herein without departing from the spirit and scope of the invention. Accordingly, it should be understood that the examples are illustrative only and are not limiting upon the scope of the invention.

### EXAMPLES

### Example 1: Overview and Materials and Methods Used in the Examples Herein

Aim of this work was to investigate the feasibility of the two constitutive phage-derived promoters T5_{N25} and T7_{A1}, recognized by the σ⁷⁰ E. coli RNAP in terms of transcription efficiency, basal expression rates and tuning capacity. The promoter sequences were modified to contain either one, two or three lacO binding sites (SEQ ID NO:28-33). The seven promoter/operator combinations that were tested with the model protein GFPmut3.1 are shown in FIG. 1. Expression strength, tunability, basal expression and cell growth were investigated in plasmid-based and plasmid-free BL21 expression systems. The resulting set of production clones was cultivated and compared under fed-batch like conditions in micro-titer fermentations.

Strains and culture conditions. Escherichia coli K-12 NEB5-α [fhuA2Δ(argF-lacZ)U169 phoA gln V44 Φ80 Δ(lacZ)M15 gyrA96 recA1 relA1 endA1 thi-1 hsdR17] was obtained from New England Biolabs (MA, USA) and used for all cloning procedures. Linear DNA cartridges were integrated into the bacterial chromosome at the attTN7 site of Escherichia coli BL21 [fhuA2 [lon] ompT gal [dcm] ΔhsdS] (New England Biolabs, MA, USA). For reference experiments, the same strains were transformed with the respective plasmids. The soluble protein GFPmut3.1 was used as recombinant model protein (19).

Basic cloning methods like restriction endonuclease (REN) digest, agarose gel electrophoresis (AGE), ligation and transformation of E. coli plasmids were carried out according to Sambrook et al. (24).

For cloning purposes, cells were routinely grown in M9ZB-medium, recovered in SOC-medium and plated on M9ZB-agar. The following antibiotic concentrations were used: ampicillin (Amp) 100 μg/ml or 30 μg/ml, kanamycin (Kan) 50 μg/ml or 30 μg/ml and chloramphenicol (Cm) 20 μg/ml or 10 μg/ml for plasmid-based and plasmid-free expression systems, respectively.

Culture Conditions

The strains were cultured in the BioLector micro-fermentation system in 48-well Flowerplates® (m2p-labs, Baesweiler, Germany) as described by Torok et al. (23). The synthetic Feed in Time (FIT) fed-batch medium with glucose and dextran as carbon sources (m2p-labs GmbH, Baesweiler, Germany) was used. Immediately prior to inoculation 0.6% (v/v) of the glucose releasing enzyme mix (EnzMix) was added. The GFPmut3.1 expression level was monitored at an excitation of 488 nm and an emission of 520 nm. The signal is given in relative fluorescence units [rfu]. The cycle time for all parameters was 20 min. The initial cell density was equivalent to an optical density of OD₆₀₀=0.3. For inoculation, a deep frozen (−80° C.) working cell bank (WCB) (OD₆₀₀=2) was thawed and biomass was harvested by centrifugation (7500 rpm, 5 min). Cells were washed with 500 μL of the corresponding medium to remove residual glycerol and centrifuged; then, pellets were re-suspended in the total cultivation medium. All cultivations were prepared in three replicates at 30° C. for 22 h. Recombinant gene expression was induced with 0.005 mM, 0.01 mM or 0.5 mM IPTG, respectively, 10 h after start of cultivation.

For fed-batch fermentations, cells were grown in a 1.5 L (1.2 L working volume, 0.4 L minimal volume) DASGIP® Parallel Bioreactor System (Eppendorf AG, DE) equipped with standard control units. The pH was maintained at 7.0±0.05 by addition of 12.5% ammonia solution (Thermo Fisher Scientific, MA/USA); the temperature was maintained at 37±0.5° C. during batch phase and was decreased to 30±0.5° C. during feed phase. The dissolved oxygen (O₂) level was stabilized above 30% saturation by controlling stirrer speed and aeration rate. Foaming was suppressed by addition of antifoam suspension (Glanapon, 2000, Bussetti, A T). For inoculation, a deep-frozen (−80° C.) working cell-bank vial was thawed and 1 ml (optical density at 600 nm=1) was transferred aseptically to the bioreactor.

Feeding was imitated when the culture, grown to 6 g cell dry mass (CDM) in 0.6 L batch medium, entered the stationary phase. A fed-batch regime with an exponential carbon-limited substrate feed was used to provide a constant growth rate of 0.1/h over 2.5 doubling times. The substrate feed was controlled by increasing the pump speed according to the exponential growth algorithm, x=x₀e^{μt}, with superimposed feedback control of weight loss in the substrate bottle. The CDW yield coefficient on glucose was 0.3 g/g and the feed medium provided glucose and components sufficient to yield an additional 32 g of CDW. Induction of the expression system was performed by adding Isopropyl-b-D-thiogalactopyranoside (IPTG) to the reactor to yield a concentration of 10 μmol/g CDW. Preparation and composition of the minimal medium used in this experiment was previously described (17).

Strains

BL21^{Q}—in Short: BQ

For the integration of the lacI^{Q} promoter in E. coli BL21 (New England BioLabs® Inc., MA/USA), the plasmid pETAmp-lacIq was constructed. This plasmid contains the ampicillin resistance gene, flanked by FRT sites and the lacI gene controlled by the lacI^{Q} promoter. The ampicillin resistance gene was amplified from pET11a using the overhang PCR technique in order to add FRT sites and the restriction sites BamHI (5′) and KpnI (3′). Following primers were used: BamHI-FRT-Amp-for and KpnI-FRT-Amp-rev.

The pBR322 ori and the lacI gene were amplified from pET30a using the overhang PCR technique in order to add a C->T mutation within the lacI promoter and the restriction sites KpnI (5′) and BamHI (3′). Following primers were used: KpnI-pBR322-for and BamHI-laciq-rev.

Linear DNA cartridges for genome integration were amplified using the Q5® High-Fidelity DNA Polymerase (New England BioLabs® Inc., MA/USA), according to the manufacturer's manual. Following primers were used: GI-lacIq-for and GI-lacIq-rev.

Integration into the bacterial chromosome occurred at the lac-operon site of E. coli BL21 (New England BioLabs® Inc., MA/USA), which carries the pSIM5 plasmid, as described by Sharan et al. (26).

Screening of positive clones and amplification of the integrated DNA cartridge was performed by basic colony PCR technique, using OneTaq® DNA Polymerase (New England BioLabs® Inc., MA/USA), according to the manufacturer's manual. Following primers were used: lacI/1_ext and laci/2_ext.

Primer AmpStop was used for sequencing the amplified DNA integration cartridge.

BL21Q::TN7<1lacOA1-GFPmut3.1-tZ>—in Short: BQ<1lacO-A1>

The sequence of the T7A1 promoter was adopted from (18) (designated as P_{A1/04}) and contains a 2 bp truncated lacO1 sequence between the −10 and −35 promoter region. This promoter was ordered as gBlocks® Gene Fragment (Integrated DNA Technologies, IA/USA), containing a 5′ spacer sequence from pET30a and the restriction sites SphI (5′) and XbaI (3′) and subsequently cloned into the pET30a-cer-tZENIT-GFPmut3.1 backbone. The new plasmid was designated as pETk1lacOA1tZ.c-GFPmut3.1.

Linear DNA cartridges for genome integration were amplified using the Q5® High-Fidelity DNA Polymerase (New England BioLabs® Inc., MA/USA), according to the manufacturer's manual. Following primers were used: TN7_1_pET30aw/oKanR_for and TN7_2_pET30a_for.

Integration into the bacterial chromosome occurred at the attTN7 site of E. coli BL21^{Q}, which carries the pSIM5 plasmid, as described by Sharan et al. (26).

Following primers were used for screening of positive clones: TN7/1_ext and TN7/2_ext.

Primer seq_MCS-for and seq_MCS-rev were used for sequencing the amplified DNA integration cartridge.

BL21Q::TN7<1lacOT5-GFPmut3.1-tZ>—in Short: BQ<1lacO-T5>

The sequence T5_{N25} promoter was adopted from (18) and contains a 2 bp truncated lacO1 sequence between the −10 and −35 promoter region. The initial transcribed sequence (ITS) between +1 and +20 of T5_{N25} was exchanged by the ITS of T7_{A1} (21). This promoter was ordered as gBlocks® Gene Fragment (Integrated DNA Technologies, IA/USA), containing a 5′ spacer sequence from pET30a and the restriction sites SphI (5′) and XbaI (3′) and subsequently cloned into the pET30a-cer-tZENIT-GFPmut3.1 backbone. The new plasmid was designated as pETk1lacOT5tZ.c-GFPmut3.1.

BL21::TN7<2lacOA1-GFPmut3.1-tZ> and BL21::TN7<2lacOT5-GFPmut3.1-tZ>—in Short: B<2lacO-A1> and B<2lacO-T5>

Besides an increased level of lacI by the lacI^{Q} promoter, a second lacO can reduce the basal expression, by enabling DNA loop formation. For the addition of a second lacO1 sequence, 62 bp upstream of the first lacO1, an overhang PCR was performed with the templates pETk1lacOA1tZ.c-GFPmut3.1 or pETk1lacOT5tZ.c-GFPmut3.1, respectively. The forward primer (2lacO-for) contains the lac-operator and the restriction site SphI (5′), the reverse primer (2lacO-rev) contains the restriction site NdeI (3′). The new plasmids were designated as pETk2lacOA1tZ.c-GFPmut3.1 and pETk2lacOT5tZ.c-GFPmut3.1.

Integration into the bacterial chromosome occurred at the attTN7 site of E. coli BL21 (New England BioLabs® Inc., MA/USA).

Amplification of linear DNA cartridge and screening was carried out as previously described.

Construction and Characterization of Promoter/Operator Combinations.

Basic cloning methods like restriction endonuclease (REN) digest, agarose gel electrophoresis (AGE), ligation and transformation of E. coli plasmids were carried out according to Sambrook et al. (24). For the integration of the lacI^{Q} promoter in E. coli BL21 (New England BioLabs® Inc., MA/USA), the plasmid pETAmp-lacIq was constructed. This plasmid contains the ampicillin resistance gene, flanked by FRT sites and the lacI gene controlled by the lacI^{Q} promoter (25). The pBR322 ori and the lacI gene were amplified from pET30a using the overhang PCR technique in order to add a C->T mutation within the lacI promoter. The linear lacI^{Q} DNA cartridge for genome integration was amplified using the Q5® High-Fidelity DNA Polymerase (New England BioLabs® Inc., MA/USA), according to the manufacturer's manual. Integration into the bacterial chromosome occurred at the lac-operon site of E. coli BL21, which carries the pSIM5 plasmid, as described by Sharan et al. (26). This strain got the designation BL21^{Q}. The sequences of the T7A1 and the T5N25 promoter were adopted from Lanzer and Bujard (18) (designated as P_{A1/04} and P_{N25/04}) and contain a 2 bp truncated lacO1 sequence between the −10 and −35 promoter region. These promoters were ordered as gBlocks® Gene Fragments (Integrated DNA Technologies, IA/USA), containing a 5′ spacer sequence from pET30a and the restriction sites SphI (5′) and XbaI (3′) and subsequently cloned into the pET30a-cer-tZENIT-GFPmut3.1 backbone. The tZENIT terminator is described elsewhere (27). A second lacO1 sequence, 62 bp upstream of the first lacO1, was added via overhang PCR. The 3lacO-T5 promoter/operator combination was adopted from pJexpress 401-406 (T5) vector from ATUM (CA/USA). Linear DNA cartridges were integrated into the bacterial chromosome at the attTN7 site of E. coli BL21 or BL21^{Q}.

GFPmut3.1 Off-Line Expression Analysis and Quantification

Recombinant GFPmut3.1 was quantified by ELISA according to Reischer et al. (28). SDS-PAGE analysis was performed as previously described (29).

Flow Cytometry

A Gallios flow cytometer (Beckman Coulter, CA/USA) was used to determine the fraction of GFPmut3.1-producing cells. Cells were harvested 12h after induction and then diluted 1/2025 in PBS. Excitation of GFPmut3.1 fluorescence was performed using an OPSL Sapphire Laser at 488 nm, with subsequent emission being measured through use of the FL1 Channel (505-545). Data were recorded for 15000 cells per sample at 300 events/sec and analyzed with Kaluza analysis software (Beckman Coulter).

LacI Western Blot and Quantification

Cell extracts obtained from ˜1.2×10⁷ BL21-wt and B<2lacO-A1> cells were separated by SDS-PAGE as previously described (29). After separation, the proteins were blotted on the provided membrane using the iBlot® Dry Blotting System according to the manufacture's manual (Invitrogen™/Thermo Fisher Scientific, CA/USA). Subsequently, proteins were blocked 4 hours at room temperature with 3% nonfat dry milk in PBST (1×PBS Dulbecco and 0.05% Tween 20). The blot was then incubated with primary antibody (1:1000 anti-LacI Antibody, clone 9A5 (Sigma-Adrich/Merck, MO/USA) 1 hour at room temperature. It was then incubated with alkaline phosphatase conjugated secondary antibody (1:2000 Anti-Mouse IgG (whole molecule)—Sigma A5153 (Sigma-Adrich/Merck, MO/USA) for 1 hour at room temperature and developed with SigmaFAST™ BCIP®/NPT tablets (Sigma-Adrich/Merck, MO/USA) according to the manufacturer's manual. Band intensities were quantified with ImageQuant TL software (GE Healthcare, IL/USA).

TABLE 1
Primers used in the Examples.
Underlined: binding part of overhang primers,
italic: overhang,
bold uppercase letters: restriction sites,
lowercase letters: lacI^{Q},
bold lower-case letter: FRT-sites,
underlined bold uppercase: C->T mutation
in lacI^{Q} promoter.

| Name | Sequence 5′-3′ |
|---|---|
| BamHI-FRT- | CAAGTCG [symbol] CGAT [symbol] |
| Amp-for | [symbol] [symbol] |
| | [symbol] AGAAAAAAAGGATC |
| | TCAAGAAG |
| | (SEQ ID NO: 7) |
| KpnI-FRT- | ACGGGGTCG [symbol] CCT |
| Amp-rev | [symbol] [symbol] [symbol] |
| | [symbol] [symbol] GTTA |
| | GCAATTTAACTGTGATAAAC |
| | (SEQ ID NO: 8) |
| KpnI- | AGGG [symbol] C GACCCCGTAGAAA |
| pBR322- | AGATCAAAGGATC |
| for | (SEQ ID NO: 9) |
| BamHI-laciq- | ATCG [symbol] CGACATCCCGGACAC |
| rev | CATCGAATGGTGCAAAAC |
| | (SEQ ID NO: 10) |
| GI-lacIq-for | CGTTACTGGTTTCACATTCACCAC |
| | (SEQ ID NO: 11) |
| GI-lacIq-rev | CGCAGGCTATTCTGGTGGCCGGAAG |
| | GCGAAGCGGCATGCATTTACGTTGA |
| | CCTTTGATCTTTTCTACGGGGTCGG |
| | (SEQ ID NO: 12) |
| lacI/1_ext | CGTAAAAATGCGCTCAGGTCAAATT |
| | CAG |
| | (SEQ ID NO: 13) |
| laci/2_ext | CAGATCGAAGAAGGGGTTGAATCGC |
| | (SEQ ID NO: 14) |
| AmpStop | TCAGGCAACTATGGATGAAC |
| | (SEQ ID NO: 15) |
| TN7_1_ | AGATGACGGTTTGTCACATGGAGTT |
| pET30aw/ | GGCAGGATGTTTGATTAAAAACATA |
| oKan R_for | GTAGTAGGTTGAGGCCGTTG |
| | (SEQ ID NO: 16) |
| TN7_2_ | CAGCCGCGTAACCTGGCAAAATCGG |
| pET30a_ | TTACGGTTGAGTAATAAATGGATGC |
| for | GAAGATCCTTTGATCTTTTCTACG |
| | (SEQ ID NO: 17) |
| TN7/1_ext | ACCGGCGCAGGGAAGG |
| | (SEQ ID NO: 18) |
| TN7/2_ext | TGGCGCTAATTGATGCCG |
| | (SEQ ID NO: 19) |
| 2lacO-for | GT [symbol] TACACGTACTTAGTC |
| | GCTGAAaattgtgagcggataaca |
| | att CCATACCCACGCCGAAA |
| | (SEQ ID NO: 20) |
| 2lacO-rev | CTTTGCTCATATGTATATCTCCTTC |
| | (SEQ ID NO: 21) |
| seq_MCS-for | GTAGTAGGTTGAGGCCGTTG |
| | (SEQ ID NO: 22) |
| seq_MCS-rev | CGGATATAGTTCCTCCTTTCAG |
| | (SEQ ID NO: 23) |

TABLE 2
gBlocks® Gene Fragments used
in the Examples. bold uppercase letters:
restriction sites, bold and italic: −35
and −10 region, underlined: lacO1*, bold
lowercase letters: native ITS of
T7_{A1} promoter.

| Name | Sequence 5′-3′ |
|---|---|
| T5A1 | GAATGGTGCATGCAAGGAGATGGC |
| | GCCCAACAGTCCCCCGGCCACGGG |
| | GCCTGCCACCATACCCACGCCGAA |
| | ACAAGATCATAAAAAATTTAT [symbol] |
| | [symbol] TGTGAGCGGATAACAAT [symbol] |
| | AGATTCatcgagagggacacggcgaa |
| | ctctagaACGGATATAGTCCTTCAG |
| | (SEQ ID NO: 24) |
| A1A1 | GAATGGTGCATGCAAGGAGATGGCGC |
| | CCAACAGTCCCCCGGCCACGGGGCCT |
| | GCCACCATACCCACGCCGAAACAAGT |
| | TTATCAAAAAGAGTG [symbol] [symbol] TGTG |
| | AGCGGATAACAAT [symbol] TAGATTC |
| | atcgagagggacacggcgaactctag |
| | aACGGATATAGTCCTTCAG |
| | (SEQ ID NO: 25) |

Table 3. Promoter sequences used in the Examples. Promoter sequences were cloned into pET30a-cer plasmid via SphI and NdeI restriction sites. Italic upper-case letters: restriction sites, lower case letters: lac operators, underlined: core promoter sequence, italic bold upper-case letters: −35 and −10 promoter elements, italic bold lower case letters: ribosomal binding site, bold upper case letters: +1 T7A1+20 initial transcribed sequence.

| Name | Sequence 5′-3′ |
|---|---|
| 3lacO-T5 | GCATGC TTACACGTACTTAGTCGCTGAA |
| | aattgtgagcggataacaattACGAGCTT |
| | CATGCACAGTTAA ATCATAAAAAATTTAT |
| | [symbol] tgtgagcggataacaat [symbol] |
| | A tgtggaattgtgagcgctcacaattcca |
| | caACGGTTTCCCTCTAGAAATAATTTTGTT |
| | TAACTTTAAG [symbol] ATATACATATG |
| | (SEQ ID NO: 28) |
| 2lacO-T5 | GCATGC TTACACGTACTTAGTCGCTGAA |
| | aattgtgagcggataacaattCCATACCC |
| | ACGCCGAAACAAG ATCATAAAAAATTTAT |
| | [symbol] tgtgagcggataacaat [symbol] |
| | AGATTC ATCGAGAGGGACACGGCGAA |
| | CTCTAGAAATAATTTTGTTTAACTTTAAG |
| | [symbol] ATATA CATATG |
| | (SEQ ID NO: 29) |
| 1lacO-T5 | GCATGCAAGGAGATGGCGCCCAACA |
| | GTCCCCCGGCCACGGGGCCTGC |
| | CACCATACCCACGCCGAAACAAG |
| | ATCATAAAAAATTTAT [symbol] |
| | tgtgagcggataacaat [symbol] |
| | AGATTC ATCGAGAGGGACACGGCGAA |
| | CTCTAGAAATAATTTTGTTTAACTTTAAG |
| | [symbol] ATATA CATATG |
| | (SEQ ID NO: 30) |
| 2lacO-A1 | GCATGC TTACACGTACTTAGTCGCTGAA |
| | aattgtgagcggataacaatt |
| | CCATACCCACGCCGAAACAAG |
| | ATCATAAAAAAGAGTG [symbol] |
| | tgtgagcggataacaat [symbol] |
| | TGATTC ATCGAGAGGGACACGGCGAA |
| | CTCTAGAAATAATTTTGTTTAACTTTA |
| | AG [symbol] ATATA CATATG |
| | (SEQ ID NO: 31) |
| 1lacO-A1 | GCATGCAAGGAGATGGCGCCCAACAGT |
| | CCCCCGGCCACGGGGCCTGC |
| | CACCATACCCACGCCGAAACAAG |
| | TTTATCAAAAAGAGTG [symbol] |
| | tgtgagcggataacaat [symbol] |
| | TAGATTC ATCGAGAGGGACACGGCG |
| | AACTCTAGAAATAATTTTGTTTAACT |
| | TTAAG [symbol] ATATA CATATG |
| | (SEQ ID NO: 32) |
| T7 | GCATGCAAGGAGATGGCGCCCAAC |
| | AGTCCCCCGGCCACGGGGCCTGC |
| | CACCATACCCACGCCGAAACAAGC |
| | GCTCATGAGCCCGAAGTGGCGAGC |
| | CCGATCTTCCCCATCGGTGATGTC |
| | GGCGATATAGGCGCCAGCAACCGC |
| | ACCTGTGGCGCCGGTGATGCCGGC |
| | CACGATGCGTCCGGCGTAGAGGA |
| | TCGAGATCGATCTCGATCCCGCGA |
| | AAT TAATACGACTCACTATAGG |
| | ggaattgtgagcggataacaattc |
| | c CCTCTAGAAATAATTTTGTTTA |
| | ACTTTAAG [symbol] ATATA CATATG |
| | (SEQ ID NO: 33) |

### Example 2: Productivity of Host RNAP Dependent Promoters/Operator Combinations

The T7 expression system is known to provide high expression rates, even from a single target gene copy, integrated into the E. coli genome. First it was tested whether the same productivity can be reached by σ⁷⁰ E. coli RNAP dependent promoters in the same experimental set-up. Therefore, plasmid-free and plasmid-based T5_{N25} and T7_{A1} promoter/operator combinations were compared with the T7 expression system. The cells were grown in fed-batch like conditions in micro-titer fermentations over a period of 22 hours. Expression of GFP was induced by a single pulse of IPTG of 0.5 mmol/L after 10 hours.

In all promoter/operator combinations, the cells were able to maintain growth during the production period of 12 hours in the micro-titer fermentations. An average growth rate of μ=0.05 h⁻¹ allowed for direct comparison of the T7 and the host RNAP dependent promoters.

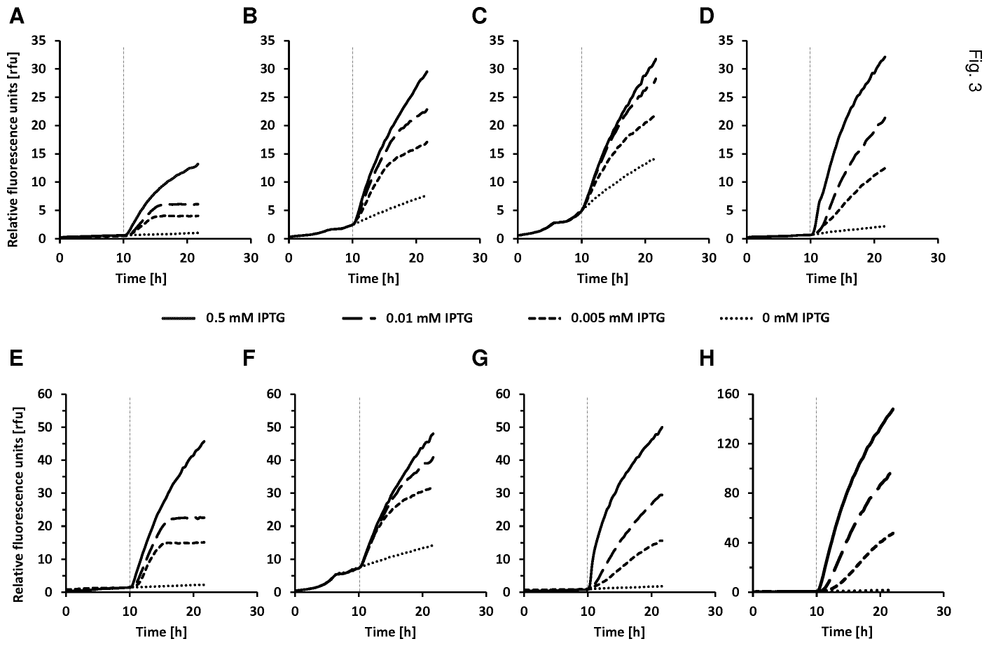

In plasmid-based expression systems, results from on-line fluorescence measurements of GFPmut3.1 were in a similar range as the T7 expression system for all promoter/operator combinations, except for B(3lacO-T5). (FIG. 2B). These results were confirmed by SDS-PAGE analyses. However, in genome-integrated expression systems, quite distinctive differences of the respective promoter/operator combinations could be observed (FIG. 2A). As compared to the T5 expression systems, GFPmut3.1 yields were 1.5-fold higher in the A1 expression systems. In the genome-integrated T7 expression system, induction of GFP gene expression led to 145 rfu and a specific product concentration (Y_{P/X}) of ˜135 mg/g soluble GFPmut3.1 and negligible amounts inclusion bodies (IBs). The same experiment with the A1 expression systems yielded almost 50 rfu and 37 mg/g soluble GFPmut3.1 without IBs.

The observed reduced productivity of B(3lacO-T5) and B<3lacO-T5> may result from the perfectly symmetric lac-operator (sym-lacO) (7) at the initial transcribed sequence (ITS) which has an influence on promoter escape and therefore, productivity (21). This effect was less visible in the plasmid-based 3lacO-T5 expression system, where the high plasmid copy number compensates for the reduced promoter activity. However, since in the plasmid-free expression system, the promoter activity was quite low, the three lacO version was dismissed for the A1 promoter. For one and two lacO promoter/operator combinations, the sym-lacO was replaced by the native ITS of the A1 promoter (+1-+20). This resulted in a 2.4-fold increase in productivity in case of the T5 promoter. However, a reduction in lacO binding sites leads inevitably to increased basal expression.

### Example 3: Basal Expression in Host RNAP Dependent Expression Systems

For challenging proteins even low basal expression can have adverse effects on host metabolism. Sometimes transformation of plasmids or integration cartridges lead to toxicity and it is difficult to obtain transformants. Therefore, tightness of gene regulation is an important quality criterion of expression systems.

In plasmid-based systems, promoters that were controlled by one lac-operator (1lacO) showed the highest basal expression at a level of ˜10 rfu, especially under C-limited conditions. The addition of a second lacO (2lacO) or the increase of the inhibitor LacI by introducing the lacI^{Q} promoter reduced the basal expression of the A1 promoter to 50%. In case of the T5 promoter, only the combination of three lac-operators (3lacO) reduced basal expression to almost 0 rfu. In contrast to the plasmid-based expression systems, in all genome integrated systems a significant impact of the promoter/operator combination on systems leakiness could be observed. Both, the increase of LacI molecules or the addition of a second lacO reduced the basal expression of A1 expression systems from 14 rfu to nearly no significant background expression and without reduction in productivity (FIG. 2A). Although, both promoters contain the lac-operators in the identical position, only an increased level of LacI molecules or three lac-operators reduced basal expression of T5 expression systems sufficiently. The T7_{A1} promoter is recognized by RNAP only half as efficiently as T5_{N25} (20) and as one lac-operator is located within the promoter sequence between the −10 and −35 promoter elements, host RNAP and lac-repressor compete each other for their respective binding site which determines how efficiently promoter activity is controlled by repressors.

### Example 4: Control of Recombinant Gene Expression Rate

Transcription rate control, also referred to as fine-tuning of protein production or “tunability” is highly relevant in bioprocessing. Optimal bioprocesses are designed to maximally exploit the cells' synthesizing capacity during a maximal long period, yielding proper folded and processed protein. Depending on the physical properties and metabolic requirements of the desired product, the transcription rates must be adapted, to be in accordance with RNA stability, translation efficiency, folding, transport an all other interactions within the system.

To evaluate the tunability of the promoter/operator combination described herein, a series of fed-batch like microtiter cultures at varying IPTG levels were tested and compared to the plasmid-free T7 expression system. Induction was performed using a single pulse of 0.005, 0.01 and 0.5 mM IPTG. On-line fluorescence measurement and end-point flow cytometry analysis were used to characterize the different promoter/operator combinations.

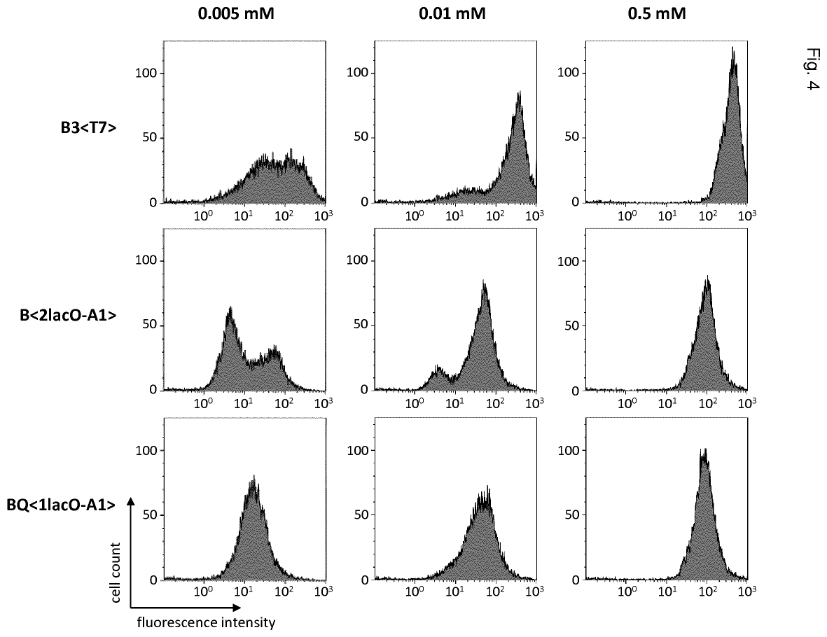

Expression systems, controlled by one lacO for gene regulation, exhibited not only the highest basal expression but also the least pronounced graduation of GFPmut3.1 expression at the given inducer concentrations (FIG. 3C, F). Although, promoters with two lacOs showed sufficiently low basal expression, they produced significantly less at lower inducer concentrations (FIG. 3B, E). The promoter/operator combinations 3lacO-T5 and 2lacO-A1 lead to a complete production stop of recombinant GFP after a certain time, independently of inducer concentration (FIG. 3 A, E). This behavior was not observed in promoter/operator combinations with only one lacO. Promoters controlled by one lacO, the lacI^{Q} (FIG. 3D, G) and the T7 expression system (FIG. 3H) combine the desired properties of low systems leakiness and tunability.

However, the T7 expression system is known to exhibit an “all-or-none” behavior, where the reduced expression level in partially induced cultures is the result of the formation of subpopulations of fully induced and non-induced cells, as reviewed in (22). To answer the question, if single-cell tunability in host RNAP dependent expression systems is possible, flow cytometry analysis of all promoter/operator combinations was performed. As shown in FIG. 4, the genome-integrated T7 expression system exhibits no homogeneous population in partially induced cultures. In fact, a mixture of fully, partially and not induced cells was found particularly at very low inducer concentrations. In the B<2lacO-A1> expression system, the flow cytometry analysis revealed two distinct sub-populations of producing and non-producing cells (FIG. 4), as these expression systems stopped their productivity, but still continued to grow. This behavior was also observed in B<3lacO-T5>. This was different for BQ<1lacO-A1>, where the induction of GFP resulted in homogenous populations at any given IPTG concentration (FIG. 4).

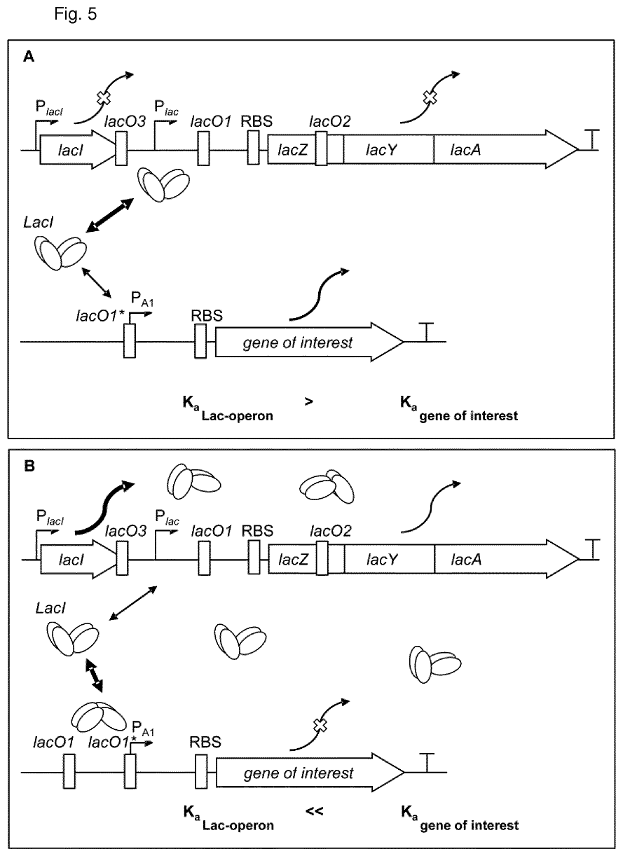

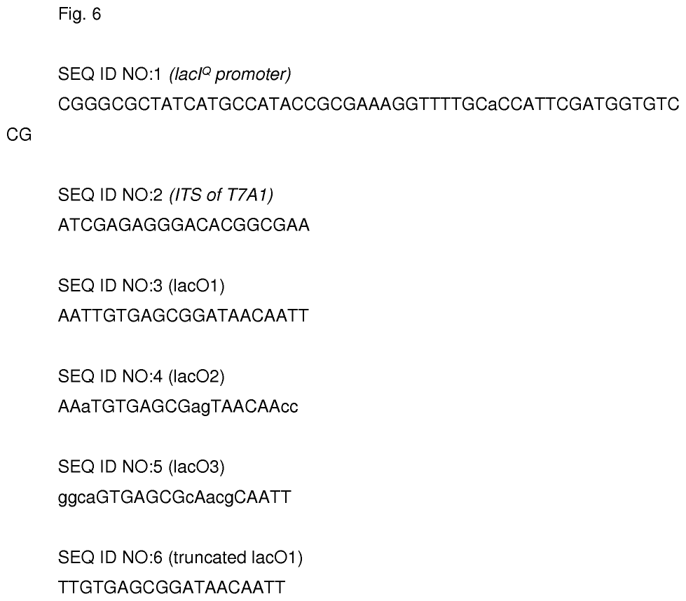

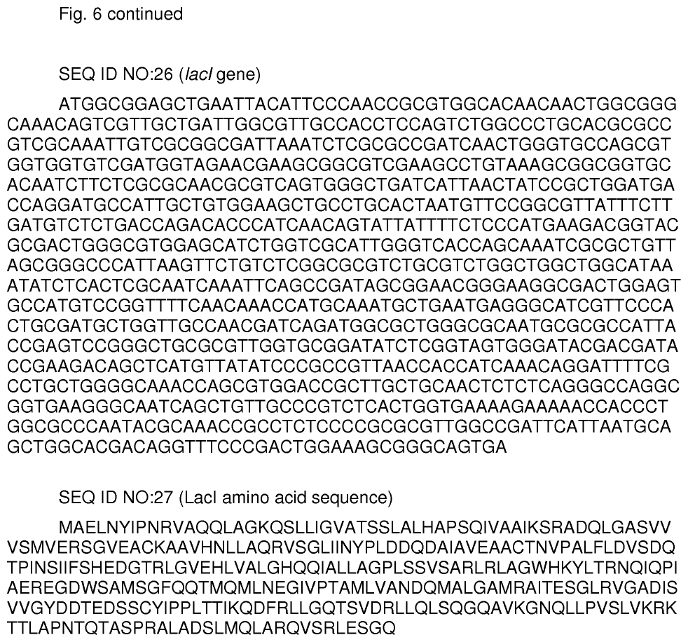

Based on these findings, it appears that the complete stop in productivity of all other expression systems when partially induced is associated with the autoregulation of the lac inhibitor. The lac-operon is regulated by 3 lacO binding sites (FIG. 5A). The LacI molecule binds to either lacO1 and lacO3 or lacO1 and lacO2. LacO3 overlaps with the 3′ end of the lacI gene. The binding of LacI to lacO1 and lacO3 causes a loop formation of the DNA and results in truncated lacI mRNA molecules, which are digested by the cell. This results in a constant level of ˜40 molecules in fully induced cells and ˜15 molecules in non-induced cells.

If the binding constant (Kₐ) of LacI to lacO at the gene of interest (GOI) is higher than the binding constant to the lacO at the lac-operon, the first LacI molecules, which are not inactivated by IPTG will preferentially bind to the lacO binding sites of the GOI instead of the lacO3/lacO1 on the lac-operon. Hence, autoregulation of LacI does not intervene and more LacI molecules are being produced (FIG. 5B). The whole system becomes over regulated and results in a complete stop in production.

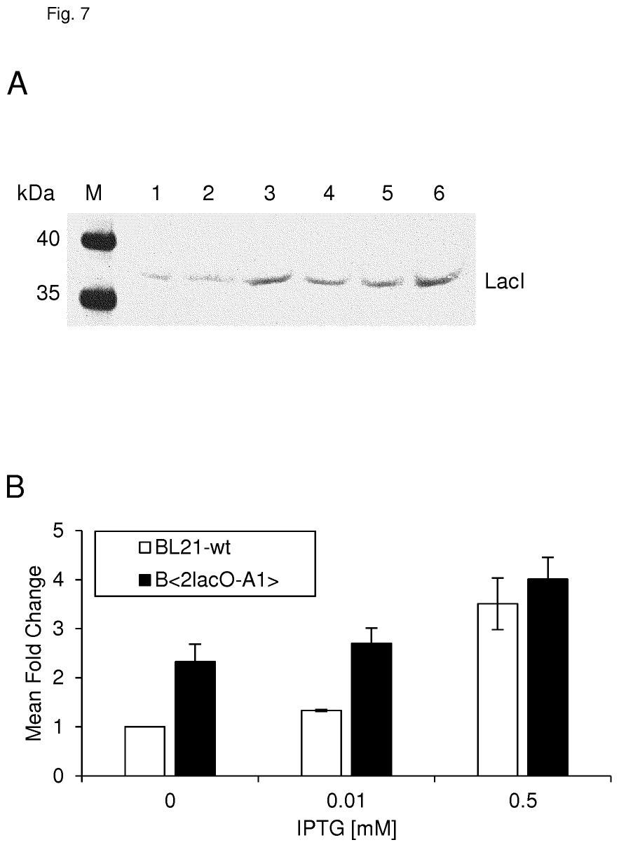

To support this hypothesis, the effect of autoregulation on LacI levels of B<2lacO-A1> and BL21 wild-type (BL21-wt) cells was compared. The LacI content of non-induced, partially and fully induced cells was estimated using western blot analysis. The band intensities were quantified and normalized with the cell number (FIG. 7).

In fully induced BL21 wild-type cells, the amount of LacI molecules was 3.5-fold, compared to non-induced BL21 wild-type cells. Partially induction with 0.01 mM IPTG only led to a 0.3-fold increase. The fold change of 3.5 in fully induced BL21-wt cells is in accordance with the results of Semsey et al., who measured on average 15 LacI molecules per cell in the absence of inducer and ˜40 molecules in fully induced cells (11). In B<2lacO-A1>, LacI amounts of non-induced and partially induced cells were clearly higher com-pared to BL21 wild-type. LacI yields were 2.3-fold in the absence of inducer and 2.7-fold in partially induced cells relative to BL21-wt. In fully induced cells, LacI yields were 4.0-fold, which corresponds with the fully induced wild-type BL21.

Although the addition of 0.01 mM IPTG results in almost half-maximal GFPmut3.1 expression (FIG. 3), it has almost no influence on LacI levels. Obviously, LacI is still able to bind to lacO1/lacO3 in the lac operon, hence maintaining its autoregulation under these conditions. In addition to that, the lacI gene is transcribed from a weak promoter resulting in about one new mRNA per cell generation (38), unlike the strong T7A1 promoter. Yet, the high LacI levels in non-induced and partially induced B<2lacO-A1>cells clearly support our hypothesis of the impact of LacI autoregulation on expression rate control in genome-integrated E. coli production strains as depicted in (Figure).

The effect of LacI autoregulation was only observed in genome-integrated host RNAP dependent expression systems, which are controlled by two or three lac operators. However, this effect was not observed in plasmid-based host RNAP dependent expression systems or in the conventional T7 expression system. The reason for this can be seen in the balance of lac operators to LacI concentration. The T7 expression system harbors a further lacI gene sequence within its DE3 lysogen, thus theoretically a doubling of the LacI concentration per cell. The plasmid-based expression systems used in this work are based on the pET plasmid system that encode a further lacI gene sequence. That in turn results in further 15-20 lacI gene sequences, depending on the plasmid copy number. However, the effect of LacI autoregulation on partially induced cells can also be observed in plasmid-based expression systems as seen in the case of E. coli pAVEway™ expression system from Fujifilm Diosynth Biotechnologies (NC/USA). In this plasmid-based expression system, transcription control is enabled by two perfectly symmetric lac operators, one positioned upstream of the T7A3 promoter and one downstream. The high affinity of LacI to the symmetric lac operators combined with the ability of DNA loop formation results in very low basal expression but exhibits also a complete stop in productivity in partially induced cultures.

Considering the autoregulation of the lac-inhibitor, a promoter/operator combination, which fulfils the desired properties such as high expression rate, negligible basal expression and true control of expression rate even at low inductor concentrations without a complete stop of productivity could successfully be identified.

Conclusion

The regulation of transcription in E. coli is receiving considerable attention because it is the first step in the process of recombinant protein production. Transcription control allows a cell to assign its resources towards the production of the recombinant protein and a tight and tunable control is essential for successful bioprocesses. It is evidenced herein that in plasmid-free expression systems, the regulatory elements of the lac-operon must be well balanced to control host RNAP dependent promoters. Three lac-operators reduce basal-expression to negligible amounts, but also the recombination production rate. The perfectly symmetric lacO in the initial transcribed sequence (ITS) hampers promoter escape of the RNAP. As shown by Hsu et al., the wild-type ITS of T7A1 exhibits an enrichment of purines and one of the best promoter escape properties (21)

Promoters containing only one lacO exhibit considerable higher promoter strength, but also higher systems leakiness. In promoter/operator combinations containing two lacOs, the two lacO1 in a distance of 62 bp at the site of the GOI exhibit a very strong binding affinity to the repressor molecule and thus prevent lacI autoregulation which results in a complete stop in productivity in partially induced cells. However, the binding affinity can be reduced by the use of less symmetric lacOs like lacO3 or lacO2 or by varying the distance between them (see Example 5).

As demonstrated herein, the combination of one lacO with an increased level of intracellular LacI caused by the lacI^{Q} promoter results in high expression rates, low basal expression and true tunability on a cellular level. Thus, this novel expression system is specifically suitable for the production of challenging proteins, as there is no plasmid-mediated metabolic load and by using the host RNAP the genetic stability increases.

Importantly, the inducible system described herein demonstrates significantly improved expression rates, reduced basal expression and true tunability compared to the T7 expression system (see e.g. FIGS. 3 and 4). The inducible expression system described herein fulfills all desired properties that are required for an efficient expression system, such as high expression rate, negligible basal expression and true control of expression rate that is steplessly adjustable, even at low inducer concentrations.

### Example 5: Control of Recombinant Gene Expression Rate in an Inducible Expression System Comprising Two lacOs

Strains: BL21::TN7<2lacO.xxA1-GFPmut3.1-tZ> and BL21::TN7<2lacO.xxT5-GFPmut3.1-tZ>—in short: B<2lacO.xx-A1> and B<2lacO.xx-T5>

For the addition of a second lacO1 sequence at a bigger distance to the first lacO1 than 62 bp, an overhang PCR is performed using the templates pETk1lacOA1tZ.c-GFPmut3.1 or pETk1lacOT5tZ.c-GFPmut3.1, respectively. The two lacO1 operators are 92, 103, 114 or 125 bp apart. The forward primers 2lacO.92-for, 2lacO.103-for, 2lacO.114-for and 2lacO.125-for contain the lac-operator and the restriction site SphI (5′), the reverse primer (2lacO-rev) contains the restriction site NdeI (3′). The new plasmids are designated as pETk2lacO.92A1tZ.c-GFPmut3.1, pETk2lacO.103A1tZ.c-GFPmut3.1, pETk2lacO.114A1tZ.c-GFPmut3.1, pETk2lacO.125A1tZ.c-GFPmut3.1 and pETk2lacO.92T5tZ.c-GFPmut3.1, pETk2lacO.103T5tZ.c-GFPmut3.1, pETk2lacO.114T5tZ.c-GFPmut3.1, pETk2lacO.125T5tZ.c-GFPmut3.1.

Integration into the bacterial chromosome occurs at the attTN7 site of E. coli BL21 (New England BioLabs® Inc., MA/USA).

Amplification of linear DNA cartridge and screening is carried out as described above.

### Example 6: Fab Production Using BQ<1lacO-A1> in Fed-Batch Culture

The T7 based expression system shows a unique strength sufficient for high expression rates even from a single copy. For systems with a single copy of the GOI under control of a host RNAP specific promotor significantly decreased expression rates are expected. Consequently, such systems will not be competitive in case when recombinant proteins must be produced at high levels. The situation is different for antibody fragments and other challenging proteins where the final product yield is definitely not determined by the strength of the promoter system but by currently un-identified reasons. To investigate these aspects, the BQ<1lacO-A1> expression system was selected for the production of the leader/Fab combination dsbA/FTN2 (dFTN2) and was compared with B3<T7> producing the same leader/Fab combination. The cells were grown in fed-batch mode at a constant growth rate of 0.1/h feed of defined medium. In the experiment the amount of cell dry weight to be produced is pre-defined to 40 g CDW. Recombinant gene expression was induced by single pulse of IPTG of 10 μmol/gCDW at 0.5 doublings past feed start.

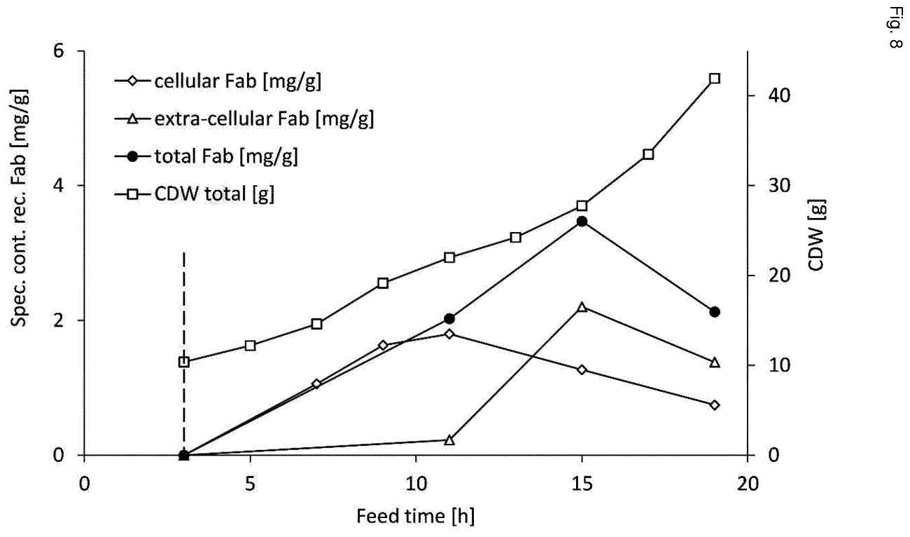

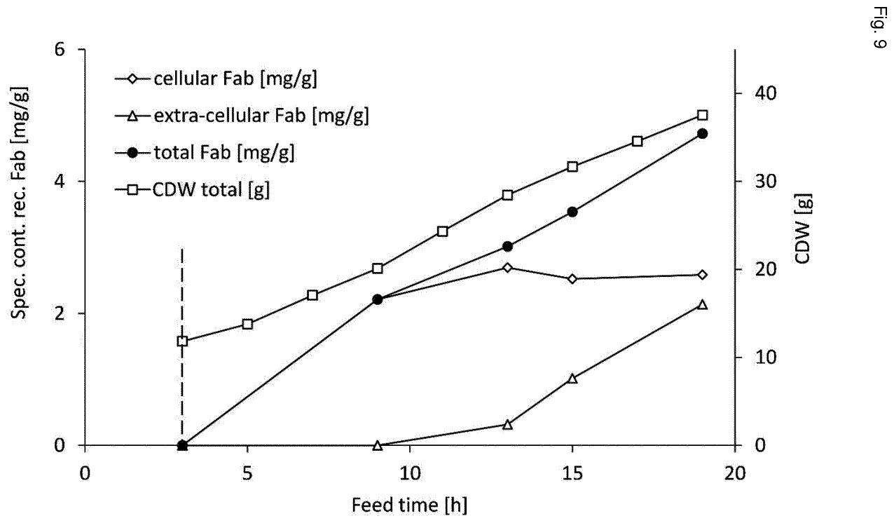

The results in FIG. 8 and FIG. 9 are given in total specific content of recombinant Fab per cell dry weight (mg/g), which is the sum of extra-cellular Fab measured in the fermentation supernatant and cellular Fab. In the T7-based system (FIG. 8), induction of dFTN2 expression led to a maximum cellular Fab concentration of 1.8 mg/g 11 hours after induction and dropped to 0.7 mg/g at end of fermentation (FIG. 8, open diamonds). At this time period, extra-cellular Fab increased from almost 0 mg/g to 2.2 mg/g (FIG. 8, open triangles). This results in a maximum total Fab concentration of 3.5 mg/g 15 hours after induction which dropped to 2.1 mg/g at the end of fermentation (FIG. 8, black dot). The increase of extra-cellular Fab in the fermentation supernatant can be attributed to cell lysis, which could be verified by measuring the DNA content in the fermentation supernatant.

The same experiment with the BQ<1lacO-A1> expression system yielded significantly improved results (FIG. 9). The content of cellular Fab could be maintained at 2.5 mg/g during the whole fermentation (FIG. 9, open diamonds). Extra-cellular Fab content increased to 2.4 mg/g at the end of fermentation (FIG. 9, open triangle). This results in a maximum total Fab concentration of 4.7 mg/g at the end of fermentation (FIG. 9, black dot). Although the relative promoter strength of 1lacO-A1 is about 30% compared to T7, this expression system yielded the same amount of total Fab as the strong T7 expression system until 15 hours after feed start and exceeded the T7 system at the end of fermentation by factor 2. These results clearly show, that a reduced promoter strength can be beneficial for the production of challenging proteins, as it decreases the metabolic burden of the cell and stress-induced proteolysis.

### REFERENCES

1. Angius, F., Ilioaia, O., Amrani, A., Suisse, A., Rosset, L., Legrand, A., Abou-Hamdan, A., Uzan, M., Zito, F., and Miroux, B. (2018) A novel regulation mechanism of the T7 RNA polymerase based expression system improves overproduction and folding of membrane proteins, Scientific reports 8, 8572.
2. Chia-Chang Hsu, O. R. T. T. a. T. W. O. (2015) Periplasmic expression in and release of Fab fragments from Escherichia coli using stress minimization, Journal of Chemical Technology & Biotechnology 91.
3. Saida, F., Uzan, M., Odaert, B., and Bontems, F. (2006) Expression of highly toxic genes in E. coli: special strategies and genetic tools, Current protein & peptide science 7, 47-56.
4. Riggs, A. D., and Bourgeois, S. (1968) On the assay, isolation and characterization of the lac repressor, J Mol Biol 34, 361-364.
5. Barkley, M. D., Riggs, A. D., Jobe, A., and Burgeois, S. (1975) Interaction of effecting ligands with lac repressor and repressor-operator complex, Biochemistry 14, 1700-1712.
6. Oehler, S., Eismann, E. R., Kramer, H., and Muller-Hill, B. (1990) The three operators of the lac operon cooperate in repression, EMBO J 9, 973-979.
7. Sadler, J. R., Sasmor, H., and Betz, J. L. (1983) A perfectly symmetric lac operator binds the lac repressor very tightly, Proc Natl Acad Sci USA 80, 6785-6789.
8. Oehler, S., Amouyal, M., Kolkhof, P., von Wilcken-Bergmann, B., and Müller-Hill, B. (1994) Quality and position of the three lac operators of E. coli define efficiency of repression, EMBO J 13, 3348-3355.
9. Mossing, M. C., and Record, M. T., Jr. (1986) Upstream operators enhance repression of the lac promoter, Science 233, 889-892.
10. Reznikoff, W. S., Winter, R. B., and Hurley, C. K. (1974) The location of the repressor binding sites in the lac operon, Proc Natl Acad Sci USA 71, 2314-2318.
11. Semsey, S., Jauffred, L., Csiszovszki, Z., Erdossy, J., Steger, V., Hansen, S., and Krishna, S. (2013) The effect of LacI autoregulation on the performance of the lactose utilization system in Escherichia coli, Nucleic Acids Res 41, 6381-6390.
12. Rosano, G. L., and Ceccarelli, E. A. (2014) Recombinant protein expression in Escherichia coli: advances and challenges, Frontiers in microbiology 5, 172.
13. Studier, F. W., and Moffatt, B. A. (1986) Use of bacteriophage T7 RNA polymerase to direct selective high-level expression of cloned genes, J Mol Biol 189, 113-130.
14. Mairhofer, J., Scharl, T., Marisch, K., Cserjan-Puschmann, M., and Striedner, G. (2013) Comparative transcription profiling and in-depth characterization of plasmid-based and plasmid-free Escherichia coli expression systems under production conditions, Applied and environmental microbiology 79, 3802-3812.
15. Glick, B. R. (1995) Metabolic load and heterologous gene expression, Biotechnology advances 13, 247-261.
16. Marchand, I., Nicholson, A. W., and Dreyfus, M. (2001) High-level autoenhanced expression of a single-copy gene in Escherichia coli: overproduction of bacteriophage T7 protein kinase directed by T7 late genetic elements, Gene 262, 231-238.
17. Striedner, G., Pfaffenzeller, I., Markus, L., Nemecek, S., Grabherr, R., and Bayer, K. (2010) Plasmid-free T7-based Escherichia coli expression systems, Biotechnology and bioengineering 105, 786-794.
18. Lanzer, M., and Bujard, H. (1988) Promoters largely determine the efficiency of repressor action, Proc Natl Acad Sci USA 85, 8973-8977.
19. Cormack, B. P., Valdivia, R. H., and Falkow, S. (1996) FACS-optimized mutants of the green fluorescent protein (GFP), Gene 173, 33-38.
20. Deuschle, U., Kammerer, W., Gentz, R., and Bujard, H. (1986) Promoters of Escherichia coli: a hierarchy of in vivo strength indicates alternate structures, EMBO J 5, 2987-2994.
21. Hsu, L. M., Cobb, I. M., Ozmore, J. R., Khoo, M., Nahm, G., Xia, L., Bao, Y., and Ahn, C. (2006) Initial transcribed sequence mutations specifically affect promoter escape properties, Biochemistry 45, 8841-8854.
22. Marschall, L., Sagmeister, P., and Herwig, C. (2017) Tunable recombinant protein expression in E. coli: promoter systems and genetic constraints, Applied microbiology and biotechnology 101, 501-512.
23. Toeroek, C., Cserjan-Puschmann, M., Bayer, K., and Striedner, G. (2015) Fed-batch like cultivation in a micro-bioreactor: screening conditions relevant for Escherichia coli based production processes, SpringerPlus 4, 490.
24. Green, J. F. S. a. M. R. (2012) Molecular Cloning: A Laboratory Manual, Vol. 4, Cold Spring Harbor Laboratory Press, U.S.
25. Mullerhill, B., Crapo, L., and Gilbert, W. (1968) Mutants That Make More Lac Repressor, P Natl Acad Sci USA 59, 1259-+.
26. Sharan, S. K., Thomason, L. C., Kuznetsov, S. G., and Court, D. L. (2009) Recombineering: a homologous recombination-based method of genetic engineering, Nat Protoc 4, 206-223.
27. Mairhofer, J., Wittwer, A., Cserjan-Puschmann, M., and Striedner, G. (2015) Preventing T7 RNA polymerase read-through transcription-A synthetic termination signal capable of improving bioprocess stability, ACS Synth Biol 4, 265-273.
28. Reischer, H., Schotola, I., Striedner, G., Potschacher, F., and Bayer, K. (2004) Evaluation of the GFP signal and its aptitude for novel on-line monitoring strategies of recombinant fermentation processes, Journal of biotechnology 108, 115-125.
29. Laemmli, U. K. (1970) Cleavage of structural proteins during the assembly of the head of bacteriophage T4, Nature 227, 680-685.
30. Waddell et al., Tn& transposition: Recognition of the attTn7 target sequence, Proc. Natl. Acad. Sci. USA (1989), vol. 86, pp. 3958-3962.
31. Muyrers, J. P. P. Zhang, Y. and Stewart A. F. Introducing Red®/ET® Recombination: DNA Engineering for the 21 Century. Gene Cloning & Expression Technologies; 2002, edited by Michael P. Weiner & Quinn Lu, Biotechniques PRESS '02
32. Sternberg N and Hoess R. The molecular genetics of bacteriophage P1. Annu Rev. Genet. 1983; 17 123-54.
33. Gentz and Bujard, Promoters Recognized by Escherichia coli RNA Polymerase Selected by Functions: Highly Efficient Promoters from Bacteriophage T5, Journal of Bacteriology, 1985, 164(1):70-77.
34. Penumetcha et al., Improving the Lac system for synthetic biology, BIOS: A Quarterly Journal of Biology, 2010, 81(1):7-15.
35. Oehler et al., The three operators of the lac operon cooperate in repression, EMBO Journal, 1990, 9(4):973-979.
36. Müller et al., Repression of lac Promoter as a Function of Distance, Phase and Quality of an Auxiliary lac Operator, J. Mol. Biol., 1996, 257:21-29.
37. Dubendorff and Studier, Controlling Basal Expression in an Inducible T7 Expression System by Blocking the Target T7 Promoter with lac Repressor, J. Mol. Biol., 1991, 2019:45-59.
38. Hawley and McClure, Compilation and analysis of Escherichia coli promoter DNA sequences, Nucleic Acids Research, 1983, 11(8):2237-2255.

1. A genome-based expression system for production of a protein of interest (POI) in a prokaryotic host, comprising at least
a) an RNA polymerase (RNAP) gene,
b) a gene encoding a POI, comprising
a coding sequence,
a promoter operably linked to said coding sequence, wherein said promoter is recognized by the RNAP expressed from a), and
at least one lac operator (lacO) within the sequence of said promoter; and
c) a lacI gene encoding a lac repressor protein (LacI) comprising
a coding sequence,
a lacI promoter operably linked to the lad coding sequence, wherein the lad promoter is a wild-type lacI promoter or a lacI promoter which increases LacI expression;
wherein the expression rate of the POI is regulated by an inducer binding LacI.

2. The genome-based expression system of claim 1, wherein the gene encoding a POI contains (i) one lacO within the sequence of the promoter or (ii) one lacO within the sequence of the promoter and one lacO upstream of the first lacO.

3. The genome-based expression system of claim 1, wherein the gene encoding a POI contains one lacO within the sequence of the promoter, and the lad promoter is a promoter which increases LacI expression.

4. The genome-based expression system of claim 1, wherein the gene encoding a POI contains one lacO within the sequence of the promoter and one lacO upstream of the first lacO, and the lacI promoter is a promoter which increases LacI expression.

5. The genome-based expression system of claim 1, wherein the prokaryotic host is Escherichia coli (E. coli), preferably the host is E. coli of the strain BL21 or K-12.

6. The genome-based expression system of claim 1, wherein the RNAP is a heterologous or homologous RNAP, preferably the RNAP is an RNAP homologous to the host, specifically it is an E. coli RNA polymerase, preferably the σ⁷⁰ E. coli RNA polymerase.

7. The genome-based expression system of claim 1, wherein the promoter in b) of claim 1 is selected from the group consisting of T5, T5_{N25}, T7A1, T7A2, T7A3, lac, lacUV5, tac and trc.

8. The genome-based expression system of claim 1, wherein the lacI promoter which increases LacI expression is the lacI^{Q} promoter comprising SEQ ID NO:1.

9. The genome-based expression system of claim 1, wherein the lac operator is a lacO1 comprising SEQ ID NO:3, lacO2 comprising SEQ ID NO:4 or lacO3 comprising SEQ ID NO:5 or a functional variant thereof with at least 65% sequence identity or a perfectly symmetric lacO.

10. The genome-based expression system of claim 1, wherein said promoter operably linked to the coding sequence encoding the protein of interest comprises an initial transcribed sequence (ITS), preferably a native T7A1 initial transcribed sequence comprising SEQ ID NO:2.

11. The genome-based expression system of claim 1, wherein the inducer is selected from the group consisting of isopropylthiogalactoside (IPTG), lactose, methyl-β-D-thiogalactoside, phenyl-β-D-galactose and ortho-nitrophenyl-β-galactoside (ONPG).

12. The genome-based expression system of claim 1, wherein the gene encoding the POI contains one lacO1 operator within the sequence of the promoter operably linked to the coding sequence and the native T7A1 initial transcribed sequence comprising SEQ ID NO:2, and wherein the lacI promoter is a lacI^{Q} promoter.

13. The genome-based expression system of claim 1, wherein the gene encoding the POI contains two lac operators which are at least 92 or 94 base pairs (bps) apart, preferably 103, 105, 114, 116, 125, 127, 134, 136, 138 or 149 bps apart, wherein one lac operator is located within the sequence of the promoter operably linked to the coding sequence and the second lac operator is upstream of the promoter.

14. A method of plasmid-free manufacturing of a protein of interest in a prokaryotic host, using the genome-based expression system of claim 1, comprising the steps of
a) cultivating the host cells and inducing expression of the gene encoding the POI by addition of an inducer,
b) harvesting the POI,
c) isolating and purifying the POI, and optionally
d) modifying, and
e) formulating the POI.

15. An expression cassette comprising at least one heterologous gene configured to produce at least one heterologous POI, including
a) one or more coding sequences encoding the one or more POI,
b) a promoter operably linked to the one or more coding sequences, and
c) at least one lac operator (lacO) within the sequence of said promoter;
wherein the affinity of lad to lacO of c) is lower than the affinity of lad to the lac operators lacO1 and lacO3 of the endogenous lac operon of a host cell.

16. The expression cassette of claim 15, wherein the heterologous gene configured to produce at least one heterologous POI includes two lac operators, which are at least 92 or 94 bp apart, wherein one lac operator is located within the sequence of the promoter and the second lac operator is upstream of the promoter.

17. The expression cassette of claim 15, further comprising a heterologous lacI promoter, which is the lacI^{Q} promoter comprising SEQ ID NO:1 and wherein the heterologous gene configured to produce at least one heterologous POI comprises a lacO1 operator within the sequence of the promoter operably linked to the coding sequence and a native T7A1 initial transcribed sequence comprising SEQ ID NO:2.

18. A method of manufacturing of a POI in a prokaryotic host on a manufacturing scale, using the expression cassette of claim 1, comprising the steps of
a) integrating the expression cassette into the chromosome of the prokaryotic host,
b) cultivating the host cells and inducing expression of the gene encoding the POI by addition of an inducer,
c) harvesting the POI, and
d) isolating and purifying the POI, and optionally
e) modifying and
f) formulating the POI.


SEQUENCE LISTING (35 sequences)

SEQ ID NO: 1 - DNA, 53 bp, Artificial Sequence
  Promoter Sequence
cgggcgctatcatgccataccgcgaaaggttttgcaccattcgatggtgtccg

SEQ ID NO: 2 - DNA, 20 bp, Artificial Sequence
  ITS Sequence
atcgagagggacacggcgaa

SEQ ID NO: 3 - DNA, 21 bp, Artificial Sequence
  lacO1 Sequence
aattgtgagcggataacaatt

SEQ ID NO: 4 - DNA, 21 bp, Artificial Sequence
  lacO2 Sequence
aaatgtgagcgagtaacaacc

SEQ ID NO: 5 - DNA, 21 bp, Artificial Sequence
  lacO3 Sequence
ggcagtgagcgcaacgcaatt

SEQ ID NO: 6 - DNA, 19 bp, Artificial Sequence
  truncated lacO1 Sequence
ttgtgagcggataacaatt

SEQ ID NO: 7 - DNA, 72 bp, Artificial Sequence
  Primer Sequence
caagtcggatccgatgaagttcctattctctagaaagtataggaacttccagaaaaaaaggatctcaagaag

SEQ ID NO: 8 - DNA, 75 bp, Artificial Sequence
  Primer Sequence
acggggtcggtacccctgaagttcctatactttctagagaataggaacttcgttagcaatttaactgtgataaac

SEQ ID NO: 9 - DNA, 35 bp, Artificial Sequence
  Primer Sequence
aggggtaccgaccccgtagaaaagatcaaaggatc

SEQ ID NO: 10 - DNA, 41 bp, Artificial Sequence
  Primer Sequence
atcggatccgacatcccggacaccatcgaatggtgcaaaac

SEQ ID NO: 11 - DNA, 24 bp, Artificial Sequence
  Primer Sequence
cgttactggtttcacattcaccac

SEQ ID NO: 12 - DNA, 75 bp, Artificial Sequence
  Primer Sequence
cgcaggctattctggtggccggaaggcgaagcggcatgcatttacgttgacctttgatcttttctacggggtcgg

SEQ ID NO: 13 - DNA, 28 bp, Artificial Sequence
  Primer Sequence
cgtaaaaatgcgctcaggtcaaattcag

SEQ ID NO: 14 - DNA, 25 bp, Artificial Sequence
  Primer Sequence
cagatcgaagaaggggttgaatcgc

SEQ ID NO: 15 - DNA, 20 bp, Artificial Sequence
  Primer Sequence
tcaggcaactatggatgaac

SEQ ID NO: 16 - DNA, 70 bp, Artificial Sequence
  Primer Sequence
agatgacggtttgtcacatggagttggcaggatgtttgattaaaaacatagtagtaggttgaggccgttg

SEQ ID NO: 17 - DNA, 74 bp, Artificial Sequence
  Primer Sequence
cagccgcgtaacctggcaaaatcggttacggttgagtaataaatggatgcgaagatcctttgatcttttctacg

SEQ ID NO: 18 - DNA, 16 bp, Artificial Sequence
  Primer Sequence
accggcgcagggaagg

SEQ ID NO: 19 - DNA, 18 bp, Artificial Sequence
  Primer Sequence
tggcgctaattgatgccg

SEQ ID NO: 20 - DNA, 68 bp, Artificial Sequence
  Primer Sequence
gtgcatgcttacacgtacttagtcgctgaaaattgtgagcggataacaattccatacccacgccgaaa

SEQ ID NO: 21 - DNA, 25 bp, Artificial Sequence
  Primer Sequence
ctttgctcatatgtatatctccttc

SEQ ID NO: 22 - DNA, 20 bp, Artificial Sequence
  Primer Sequence
gtagtaggttgaggccgttg

SEQ ID NO: 23 - DNA, 22 bp, Artificial Sequence
  Primer Sequence
cggatatagttcctcctttcag

SEQ ID NO: 24 - DNA, 173 bp, Artificial Sequence
  Gene Fragment Sequence
gaatggtgcatgcaaggagatggcgcccaacagtcccccggccacggggcctgccaccatacccacgccgaaacaagatcataaaaaatttatttgctttgtgagcggataacaattataatagattcatcgagagggacacggcgaactctagaacggatatagtccttcag

SEQ ID NO: 25 - DNA, 174 bp, Artificial Sequence
  Gene Fragment Sequence
gaatggtgcatgcaaggagatggcgcccaacagtcccccggccacggggcctgccaccatacccacgccgaaacaagtttatcaaaaagagtgttgacttgtgagcggataacaatgatacttagattcatcgagagggacacggcgaactctagaacggatatagtccttcag

SEQ ID NO: 26 - DNA, 960 bp, Artificial Sequence
  LacI
atggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtga

SEQ ID NO: 27 - protein, 319 aa, Artificial Sequence
  LacI
MAELNYIPNRVAQQLAGKQSLLIGVATSSLALHAPSQIVAAIKSRADQLGASVVVSMVERSGVEACKAAVHNLLAQRVSGLIINYPLDDQDAIAVEAACTNVPALFLDVSDQTPINSIIFSHEDGTRLGVEHLVALGHQQIALLAGPLSSVSARLRLAGWHKYLTRNQIQPIAEREGDWSAMSGFQQTMQMLNEGIVPTAMLVANDQMALGAMRAITESGLRVGADISVVGYDDTEDSSCYIPPLTTIKQDFRLLGQTSVDRLLQLSQGQAVKGNQLLPVSLVKRKTTLAPNTQTASPRALADSLMQLARQVSRLESGQ

SEQ ID NO: 28 - DNA, 201 bp, Artificial Sequence
  Promoter
gcatgcttacacgtacttagtcgctgaaaattgtgagcggataacaattacgagcttcatgcacagttaaatcataaaaaatttatttgctttgtgagcggataacaattataatatgtggaattgtgagcgctcacaattccacaacggtttccctctagaaataattttgtttaactttaagaaggagatatacatatg

SEQ ID NO: 29 - DNA, 187 bp, Artificial Sequence
  Promoter
gcatgcttacacgtacttagtcgctgaaaattgtgagcggataacaattccatacccacgccgaaacaagatcataaaaaatttatttgctttgtgagcggataacaattataatagattcatcgagagggacacggcgaactctagaaataattttgtttaactttaagaaggagatatacatatg

SEQ ID NO: 30 - DNA, 187 bp, Artificial Sequence
  Promoter
gcatgcaaggagatggcgcccaacagtcccccggccacggggcctgccaccatacccacgccgaaacaagatcataaaaaatttatttgctttgtgagcggataacaattataatagattcatcgagagggacacggcgaactctagaaataattttgtttaactttaagaaggagatatacatatg

SEQ ID NO: 31 - DNA, 187 bp, Artificial Sequence
  Promoter
gcatgcttacacgtacttagtcgctgaaaattgtgagcggataacaattccatacccacgccgaaacaagatcataaaaaagagtgttgacttgtgagcggataacaatgatacttgattcatcgagagggacacggcgaactctagaaataattttgtttaactttaagaaggagatatacatatg

SEQ ID NO: 32 - DNA, 188 bp, Artificial Sequence
  Promoter
gcatgcaaggagatggcgcccaacagtcccccggccacggggcctgccaccatacccacgccgaaacaagtttatcaaaaagagtgttgacttgtgagcggataacaatgatacttagattcatcgagagggacacggcgaactctagaaataattttgtttaactttaagaaggagatatacatatg

SEQ ID NO: 33 - DNA, 308 bp, Artificial Sequence
  Promoter
gcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactataggggaattgtgagcggataacaattcccctctagaaataattttgtttaactttaagaaggagatatacatatg

SEQ ID NO: 34 - DNA, 6 bp, Artificial Sequence
  Consensus
tataat

SEQ ID NO: 35 - DNA, 6 bp, Artificial Sequence
  Consensus
ttgaca